(19) United States
(12) Patent Application Publication
(10) Pub. No.: US 2024/0358759 A1
(43) Pub. Date: Oct. 31, 2024

(54) COMBINATION CANCER THERAPY WITH MEMORY NK CELLS AND ANTI-CANCER BISPECIFIC MOLECULES

(71) Applicant: WUGEN, Inc., St. Louis, MO (US)

(72) Inventors: Ayman KABAKIBI, San Diego, CA (US); Ryan P. SULLIVAN, Ballwin, MO (US)

(21) Appl. No.: 18/657,954

(22) Filed: May 8, 2024

Related U.S. Application Data
(63) Continuation of application No. PCT/US2022/079528, filed on Nov. 9, 2022.
(60) Provisional application No. 63/277,505, filed on Nov. 9, 2021.

(51) Int. Cl. A61K 35/17; A61K 39/00; A61P 35/00; C07K 16/28

(52) U.S. Cl. CPC A61K 35/17; A61K 39/4613; A61P 35/00; C07K 16/2827; C07K 16/283; C07K 16/2863; C07K 2317/31

(57) ABSTRACT

Disclosed herein is method of treating cancer comprising administration of memory NK cells and a monoclonal antibody comprising a tumor antigen binding moiety and a CD16 binding moiety. Also disclosed herein is a method for treating ovarian cancer in a patient in need thereof comprising contacting ovarian cancer cells or gastric cancer cells with memory NK cells in combination with an anti-HER2 monoclonal antibody. Also disclosed herein is a method for treating head and neck cancer or colorectal cancer in a patient in need thereof comprising contacting head and neck cancer cells or colorectal cancer cells with memory NK cells in combination with an anti-EGFR monoclonal antibody. Also disclosed herein is a method for treating urothelial carcinoma, head and neck cancer, colorectal cancer, or gastric cancer cells in a patient in need thereof comprising contacting the cancer cells with memory NK cells in combination with an anti-PDL-1 monoclonal antibody.

This application is a Bypass Continuation of International Patent Application no. PCT/US2022/079528 filed on Nov. 9, 2022, which claims the benefit of priority of U.S. Provisional Patent Application No. 63/277,505, filed Nov. 9, 2021, the disclosures of which are incorporated by reference as if written herein in their entireties.

The present disclosure generally relates to, inter alia, memory/memory-like and cytokine-induced memory like (CIML) NK cells, methods of making and using them e.g. in the treatment of cancer, and increasing anti-tumor properties of NK cells by combination treatment with memory NK cells with anti-cancer monoclonal antibodies.

Natural killer (NK) cells constitute a group of innate immune cells, which are often characterized as cytotoxic lymphocytes that exhibit antibody dependent cellular toxicity via target-directed release of granzymes and perforin. Most NK cells have a specific cell surface marker profile (e.g., CD3, CD56+, CD16+, CD57+, CD8+) in addition to a collection of various activating and inhibitory receptors. While more recently NK cells have become a significant component of certain cancer treatments, generation of significant quantities of NK cells has been a significant obstacle as the fraction of NK cells in whole blood is relatively low.

Ovarian cancer is diagnosed in an estimated 300,000 people worldwide each year and causes roughly 180,000 deaths. In 2021, ovarian cancer will be diagnosed in approximately 21,000 people and cause about 14,000 deaths in the United States, where it is the leading cause of death from gynecologic cancer. The HER-2 receptor targeted monoclonal antibody trastuzumab and polyclonal NK cells show synergistic killing of naïve SKOV-3 ovarian cancer cells. However, over the course of treatment the outgrowth of therapy-resistant tumor cell clones frequently leads to relapse. There is, therefore, a need to provide more effective immunotherapies for ovarian cancer.

Gastric cancer ranks third as the most common cause of worldwide cancer specific mortality. In 2018 a total of 26,240 new cases of gastric cancer were recorded in the USA, with 10,800 deaths recorded in the same year. HER2 is a proto-oncogene belonging to the epidermal growth factor receptor (EGFR) family which promotes cell proliferation and cancer development. Expression of HER2 is observed in approximately 22-32% of gastric cancers. Trastuzumab, which targets HER2, has been evaluated for the treatment of gastric carcinoma in first-line and second-line clinical trials. Combined chemotherapy and trastuzumab in first-line clinical trials demonstrated an increased OS in patients from 11.1 to 13.8 months compared to chemotherapy only controls. However, in the recent T-ACT second-line trastuzumab combination trial no significant difference in OS survival was observed. Tumor loss and heterogeneity of HER2 remains a challenge and there remains a requirement for more effective immunotherapies in gastric cancer indications.

Colorectal cancer (CRC) has the third highest incidence rate globally with over 1.8 million new cases in 2018. In the same year 881,000 deaths were attributed to CRC globally, making this indication the second leading cause of cancer related deaths. Several CRC mutations are important in prognosis including KRAS/NRAS, BRAF, HER2, NTRK and MMR/MS which are currently recommended for molecular testing. With current treatments CRC has a 5-year relative survival rate of 64.7% in the USA however, prognosis is worse for patients with unfavorable genetic profiles. EGFR is known to be an important player in initiation and progression of colorectal carcinoma. Cetuximab improves survival and quality of life in 10-20% of patients, but many patients develop drug resistance as antibody treatment progresses. The mechanism for this resistance, at least in part, is believed to be impairment of ADCC. Therefore, developing strategies to enhance cetuximab-mediated ADCC in these patients is needed.

Head and neck cancer (HNC) is a collective term that includes several different types of cancers. Cancers of the head and neck are categorized by the area in which they begin. This includes the mouth (oral cavity), throat (pharynx), voice box (larynx), sinuses and nose cavity, and salivary glands. The EGFR-targeted monoclonal antibody is effective against HNC, but in only 15-20% of patients. Notably, cetuximab treatment increases the frequency of intratumoral Treg cells that suppress cetuximab-mediated ADCC by NK cells and their presence correlates with poor clinical outcome. There is, therefore, a need to provide more effective immunotherapies for HNC.

Urothelial carcinoma is the most common type of bladder cancer and about 50% of patients develop metastatic disease. Early-stage disease is usually treated surgically, but invasive urothelial carcinomas which have increased risk of metastasis, require other treatment approaches in conjunction to surgery. PD-L1 is a checkpoint protein expressed on many tumors that allows cells to escape the immune system by serving as a checkpoint to impair T cell function. The PD-L1 targeting checkpoint inhibitor, avelumab, has recently been approved for use in urothelial carcinoma. Patients treated with avelumab as a second-line therapy demonstrated an overall response rate of ˜17% in metastatic urothelial carcinoma. Avelumab mechanism of action also includes tumor-targeted ADCC as well as its effects on checkpoint inhibition. Notably, studies have shown that avelumab enhances NK-cell mediated toxicity against tumor targets and that tumors expressing higher levels of PD-L1 were more sensitive to avelumab-mediated ADCC. Therefore, identification of new and improved immunotherapies for urothelial carcinoma are needed.

Disclosed herein are new modalities for treatment of ovarian cancer, colorectal cancer, HNC, gastric cancer and urothelial cancer that utilize memory NK cells (sometimes called memory-like or cytokine-induced memory-like NK cells) in combination with monoclonal antibodies directed against these cancers.

### BRIEF DESCRIPTION OF THE SEQUENCES

SEQ ID NOs:1 and 2 in Table 1 are sequences of exemplary expansion fusion protein chains.

SEQ ID NOs:3 to 22 in Table 2 are sequences of exemplary anti-TF antibodies (“ATF 1”).

SEQ ID NOs:23 and 24 in Table 3 are sequences of exemplary priming fusion protein chains.

SEQ ID NOs: 25 to 60 are sequences of the heavy and light chains and VH and VL domains of certain antibodies discussed herein.

### BRIEF DESCRIPTION OF THE DRAWINGS

FIG. 1 is a schematic of experimental design for testing memory NK cells with or without monoclonal antibodies or with control non-targeting isotype antibody and the involvement of CD16.

FIG. 2 shows the results of memory NK cell cytotoxicity in conjunction with trastuzumab. Trastuzumab enhanced memory NK cell antibody-dependent cellular toxicity (ADCC) against the ovarian cancer cell line SKOV-3.

FIG. 3 shows the results of memory NK cell cytotoxicity in conjunction with cetuximab enhanced memory NK cell ADCC against the HNC cell line SCC-25.

FIG. 4 shows persistence of memory NK cells in mouse blood for at least 35 days post injection in THP-1 AML tumor model.

FIG. 5 shows increased CD16 expression on the surface of memory NK cells from the time of injection into mice and persisting throughout the course of the study.

FIG. 6 shows IFNg dose response of PD-L1 expression on SCC-25 head & neck cancer cells.

FIG. 7 shows inducement of PD-L1 expression on human LoVo colon cancer cells by IFNg.

FIG. 8 A) shows tumor cell viability and B) PD-L1 expression in colorectal cancer cell lines after treatment with memory NK cells.

FIG. 9 shows the results of memory NK cell cytotoxicity in conjunction with cetuximab, avelumab, and combinations thereof against the CRC cell line LoVo-GFP. The upper panel, A), shows PD-L1 expression on LoVo cells in the upper chamber, demonstrated as a mean fluorescent intensity (MFI), and the lower panel, B), shows PD-L1 expression on LoVo cells in the upper chamber, demonstrated as a percentage (%) of PD-L1 positive cells.

FIG. 10 shows PD-L1 induction on SCC-25 cells (head and neck cancer) after incubation with memory NK cells, as percent cytotoxicity for the primary coculture for 12 hours.

FIG. 11 shows PD-L1 induction on SCC-25 cells (head and neck cancer) after incubation with memory NK cells, as percent cytotoxicity for the primary coculture for 24 hours.

FIG. 12 shows PD-L1 induction on SCC-25 cells (head and neck cancer) after incubation with memory NK cells, as percent cytotoxicity for the primary coculture for 42 hours.

FIG. 13 shows PD-L1 induction on SCC-25 cells (head and neck cancer) after incubation with conditioned media, as percent cytotoxicity for the secondary conditioned coculture for 12 hours.

FIG. 14 shows PD-L1 induction on SCC-25 cells (head and neck cancer) after incubation with conditioned media, as percent cytotoxicity for the secondary conditioned coculture for 24 hours.

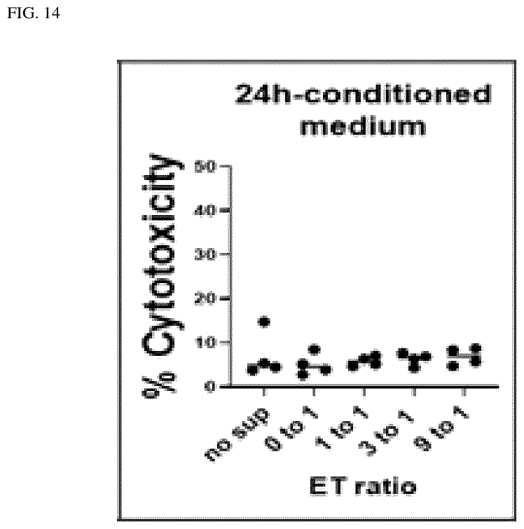

FIG. 15 shows PD-L1 induction on SCC-25 cells (head and neck cancer) after incubation with conditioned media, as percent cytotoxicity for the secondary conditioned coculture for 42 hours.

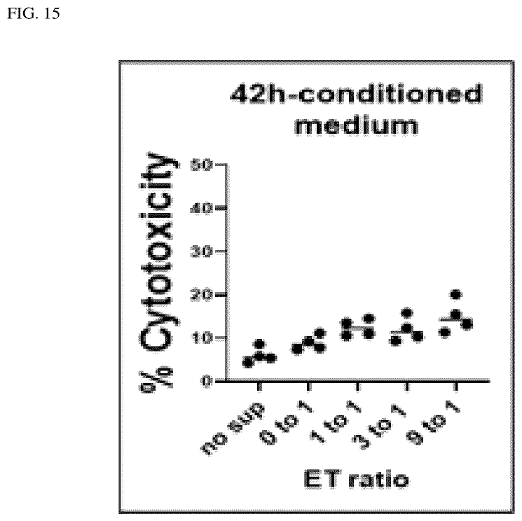

FIG. 16 shows inducement of PD-L1 on SCC-25 cells (HNC) after incubation with memory NK cells or incubation with conditioned media.

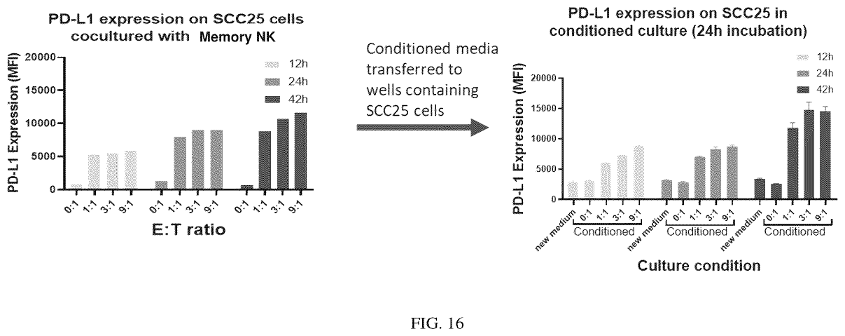

FIG. 17 shows the results of avelumab and memory NK cells cocultures for killing of NCI-N87 Cells (gastric cancer cells).

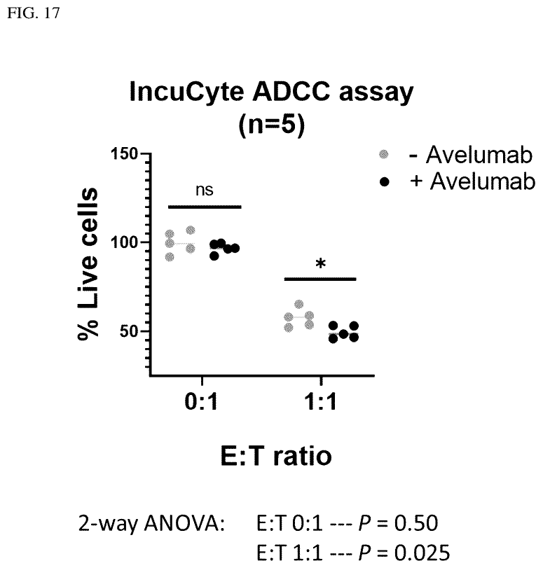

### DETAILED DESCRIPTION

Provided herein are compositions and methods that enable more effective immunotherapies against ovarian cancer, colorectal cancer, HNC, gastric cancer and urothelial cancer. The compositions include primed and expanded memory NK cells and a monoclonal antibody chosen from an anti-HER2 receptor antibody, an anti-EGFR antibody or an anti-PD-L1 antibody. The methods comprise contacting ovarian cancer cells, colorectal cancer cells, HNC cancer cells, gastric cancer cells, or urothelial cancer cells with primed and expanded memory NK cells in combination with a monoclonal antibody chosen from an anti-HER2 receptor antibody, an anti-EGFR antibody and anti-PD-L1 antibody. Memory NK cells can be generated in a process in which NK cells are concurrently primed to form the memory NK cells and expanded to a desired quantity. Alternatively, the NK cells can be expanded to a desired quantity and then primed to form the memory NK cells, or the reverse.

Provided herein is a method of treating a proliferative malignancy, the method comprising administration of the memory NK cells according to the embodiments above in combination with an anti-cancer monoclonal antibody.

In some embodiments, the proliferative malignancy is ovarian cancer and the monoclonal antibody is an anti-HER2 antibody. In some embodiments the anti-HER2 antibody is chosen from trastuzumab and margetuximab. In some embodiments the anti-HER2 antibody is trastuzumab. In some embodiments, the ovarian cancer is chosen from clear cell carcinoma, endometrioid adenocarcinoma, mucinous adenocarcinoma, serous carcinoma, stromal tumor, germ cell tumor and small cell cancer of the ovary.

In some embodiments, the proliferative malignancy is head and neck cancer (HNC) or gastric cancer and the monoclonal antibody is an anti-EGFR antibody. In some embodiments, the anti-EGFR antibody is chosen from cetuximab, panitumumab, and necitumumab. In some embodiments the anti-EGFR antibody is cetuximab. In some embodiments, the HNC is chosen from oropharyngeal cancer, hypopharyngeal cancer, laryngeal cancer, lip and oral cavity cancer, nasopharyngeal cancer, paransal sinus cancer, salivary gland cancer, squamous cell neck cancer, nasal cavity cancer, soft tissue sarcoma and thyroid cancer. In some embodiments, the gastric cancer is chosen from gastric adenocarcinomas, gastrointestinal stromal tumors, gastrointestinal neuroendocrine (Carcinoid) tumors, lymphomas, squamous cell carcinomas, small cell carcinomas and leiomyosarcomas.

In some embodiments, the proliferative malignancy is urothelial cancer and the monoclonal antibody is an anti-PD-L1 antibody. In some embodiments, the monoclonal antibody is chosen from avelumab and coseibelimab. In some embodiments, the monoclonal antibody is avelumab. In some embodiments the gastric cancer is chosen from papillary carcinoma, flat carcinoma, squamous cell carcinoma adenocarcinoma, gastric sarcoma and small cell carcinoma.

Provided herein is a method of treating cancer in a subject in need thereof comprising administering memory NK cells and a bispecific molecule (e.g., a monoclonal antibody, NK cell engager, or trispecific killer cell engager) comprising a tumor antigen binding moiety and a CD16 binding moiety.

Also provided herein is an immunotherapeutic composition comprising memory NK cells and a bispecific molecule (e.g., a monoclonal antibody, NK cell engager, or trispecific killer cell engager) comprising a tumor antigen binding moiety and a CD16 binding moiety, and a therapeutically acceptable carrier.

Also provided herein is an immunotherapeutic kit comprising memory NK cells and a bispecific molecule (e.g., a monoclonal antibody, NK cell engager, or trispecific killer cell engager) comprising a tumor antigen binding moiety and a CD16 binding moiety, and optionally, instructions to administer the memory NK cells and antibody in simultaneous or sequential combination to a subject with cancer.

In some embodiments, the bispecific molecule is a monoclonal antibody of the IgG type. In some embodiments, the monoclonal antibody is an IgG1. In some embodiments, the monoclonal antibody is an IgG3. In some embodiments, the Fc moiety of the IgG1 monoclonal antibody is mutated to enhance CD16 binding affinity.

In some embodiments, the IgG1 or IgG3 antibody is chosen from an anti-HER2 antibody, an anti-PDL-1 antibody, and an anti-EGFR antibody.

In some embodiments, the IgG1 (or IgG3) antibody is an anti-HER2 monoclonal antibody.

In some embodiments, the cancer is ovarian cancer. In some embodiments, the ovarian cancer is a clear cell carcinoma. In some embodiments, the ovarian cancer is an endometrioid adenocarcinoma. In some embodiments, the ovarian cancer is a mucinous adenocarcinoma. In some embodiments, the ovarian cancer is a serous carcinoma. In some embodiments, the ovarian cancer is a stromal tumor. In some embodiments, the ovarian cancer is a germ cell tumor. In some embodiments, the ovarian cancer is a small cell cancer of the ovary.

In some embodiments, the cancer is gastric cancer.

In some embodiments, the anti-HER2 monoclonal antibody is chosen from trastuzumab and margetuximab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies disclosed herein or known in the art. In some embodiments, the anti-HER2 monoclonal antibody is trastuzumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies disclosed herein or known in the art.

In some embodiments, the IgG1 (or IgG3) antibody is an anti-EGFR monoclonal antibody.

In some embodiments, the cancer is head and neck cancer. In some embodiments, the head and neck cancer is oropharyngeal cancer. In some embodiments, the head and neck cancer is hypopharyngeal cancer. In some embodiments, the head and neck cancer is laryngeal cancer. In some embodiments, the head and neck cancer is lip and oral cavity cancer. In some embodiments, the head and neck cancer is nasopharyngeal cancer. In some embodiments, the head and neck cancer is paranasal sinus cancer. In some embodiments, the head and neck cancer is salivary gland cancer. In some embodiments, the head and neck cancer is squamous cell neck cancer. In some embodiments, the head and neck cancer is nasal cavity cancer. In some embodiments, the head and neck cancer is soft tissue sarcoma. In some embodiments, the head and neck cancer is thyroid cancer.

In some embodiments, the cancer is colorectal cancer.

In some embodiments, the anti-EGFR monoclonal antibody is chosen from cetuximab, panitumumab, and necitumumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies disclosed herein or known in the art. In some embodiments, the anti-EGFR monoclonal antibody is cetuximab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with the antibody disclosed herein or known in the art.

In some embodiments, the IgG1 antibody is an anti-immune-checkpoint protein antibody. In some embodiments, the anti-immune-checkpoint protein antibody is chosen from an anti-PD-1 monoclonal antibody, an anti-PD-L1 monoclonal antibody, and an anti-CTLA-4 monoclonal antibody. In some embodiments, the cancer is chosen from urothelial carcinoma, head and neck cancer, colorectal cancer, and gastric cancer.

In some embodiments, the antibody is an anti-PD-L1 monoclonal antibody. In some embodiments, the anti-PD-L1 monoclonal antibody is avelumab. In some embodiments, the cancer is urothelial carcinoma. In some embodiments, the cancer is head and neck cancer. In some embodiments, the cancer is colorectal cancer. In some embodiments, the cancer is gastric cancer.

In some embodiments, the antibody is an anti-PD-1 monoclonal antibody. In some embodiments, the anti-PD-1 antibody is chosen from nivolumab, pembrolizumab, cemiplimab, and dostarlimab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies and known in the art.

In some embodiments, the antibody is an anti-CTLA-4 monoclonal antibody. In some embodiments, the anti-CTLA-4 monoclonal is antibody ipilimumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies and known in the art.

In some embodiments, the anti-PD-L1 monoclonal antibody is chosen from atezolizumab, avelumab, coseibelimab, and durvalumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies disclosed herein or known in the art. In some embodiments, the anti-PD-L1 monoclonal antibody is avelumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibody disclosed herein or known in the art.

In some embodiments, the bispecific molecule is an NK cell engager or a trispecific killer cell engager.

In some embodiments, the memory NK cells are administered before the antibody (or other bispecific molecule). In some embodiments, the memory NK cells are administered concurrently with the antibody (or other bispecific molecule). In some embodiments, the memory NK cells are administered after the antibody (or other bispecific molecule).

In some embodiments, the memory NK cells have increased expression of CD16 relative to normal NK cells. In some embodiments, the memory NK cells' increased expression of CD16 relative to normal NK cells persists for at least 30 days.

### ENUMERATED EMBODIMENTS

Embodiment 1. A method of treating cancer in a subject in need thereof comprising administering memory NK cells and a bispecific molecule (e.g., a monoclonal antibody, NK cell engager, or trispecific killer cell engager) comprising a tumor antigen binding moiety and a CD16 binding moiety.

Embodiment 2. The method of Embodiment 1, wherein the bispecific molecule is a monoclonal antibody of the IgG type.

Embodiment 3. The method of Embodiment 2, wherein the monoclonal antibody is an IgG1.

Embodiment 4. The method of Embodiment 3, wherein the Fc moiety of the IgG1 monoclonal antibody is mutated to enhance CD16 binding affinity.

Embodiment 5. The method of either of Embodiments 3 and 4, wherein the IgG1 antibody is chosen from an anti-HER2 antibody, an anti-PDL-1 antibody, and an anti-EGFR antibody.

Embodiment 6. The method of Embodiment 5, wherein the IgG1 antibody is an anti-HER2 monoclonal antibody.

Embodiment 7. The method of Embodiment 6, wherein the cancer is ovarian cancer.

Embodiment 8. The method of Embodiment 7, wherein the ovarian cancer is a clear cell carcinoma.

Embodiment 9. The method of Embodiment 7, wherein the ovarian cancer is an endometrioid adenocarcinoma.

Embodiment 10. The method of Embodiment 7, wherein the ovarian cancer is a mucinous adenocarcinoma.

Embodiment 11. The method of Embodiment 7, wherein the ovarian cancer is a serous carcinoma.

Embodiment 12. The method of Embodiment 7, wherein the ovarian cancer is a stromal tumor.

Embodiment 13. The method of Embodiment 7, wherein the ovarian cancer is a germ cell tumor.

Embodiment 14. The method of Embodiment 7, wherein the ovarian cancer is a small cell cancer of the ovary.

Embodiment 15. The method of Embodiment 6, wherein the cancer is gastric cancer.

Embodiment 16. The method of any of Embodiments 6-15, wherein the anti-HER2 monoclonal antibody is chosen from trastuzumab and margetuximab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies disclosed herein or known in the art.

Embodiment 17. The method of Embodiment 16, wherein the anti-HER2 monoclonal antibody is trastuzumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies disclosed herein or known in the art.

Embodiment 18. The method of Embodiment 5, wherein the IgG1 antibody is an anti-EGFR monoclonal antibody.

Embodiment 19. The method of Embodiment 18, wherein the cancer is head and neck cancer.

Embodiment 20. The method of Embodiment 19, wherein the head and neck cancer is oropharyngeal cancer.

Embodiment 21. The method of Embodiment 19, wherein the head and neck cancer is hypopharyngeal cancer.

Embodiment 22. The method of Embodiment 19, wherein the head and neck cancer is laryngeal cancer.

Embodiment 23. The method of Embodiment 19, wherein the head and neck cancer is lip and oral cavity cancer.

Embodiment 24. The method of Embodiment 19, wherein the head and neck cancer is nasopharyngeal cancer.

Embodiment 25. The method of Embodiment 19, wherein the head and neck cancer is paranasal sinus cancer.

Embodiment 26. The method of Embodiment 19, wherein the head and neck cancer is salivary gland cancer.

Embodiment 27. The method of Embodiment 19, wherein the head and neck cancer is squamous cell neck cancer.

Embodiment 28. The method of Embodiment 19, wherein the head and neck cancer is nasal cavity cancer.

Embodiment 29. The method of Embodiment 19, wherein the head and neck cancer is soft tissue sarcoma.

Embodiment 30. The method of Embodiment 19, wherein the head and neck cancer is thyroid cancer.

Embodiment 31. The method of Embodiment 18, wherein the cancer is colorectal cancer.

Embodiment 32. The method of any of Embodiments 19-31, wherein the anti-EGFR monoclonal antibody is chosen from cetuximab, panitumumab, and necitumumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies disclosed herein or known in the art.

Embodiment 33. The method of Embodiment 33, wherein the anti-EGFR monoclonal antibody is cetuximab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with the antibody disclosed herein or known in the art.

Embodiment 34. The method of Embodiment 5, wherein the IgG1 antibody is an anti-immune-checkpoint protein antibody.

Embodiment 35. The method of Embodiment 34, wherein the anti-immune-checkpoint protein antibody is chosen from an anti-PD-1 monoclonal antibody, an anti-PD-L1 monoclonal antibody, and an anti-CTLA-4 monoclonal antibody.

Embodiment 36. The method of Embodiment 34, wherein the cancer is chosen from urothelial carcinoma, head and neck cancer, colorectal cancer, and gastric cancer.

Embodiment 37. The method of Embodiment 36, wherein the antibody is an anti-PD-L1 monoclonal antibody.

Embodiment 38. The method of Embodiment 37, wherein the anti-PD-L1 monoclonal antibody is avelumab.

Embodiment 39. The method of either of Embodiments 37 and 38, wherein the cancer is urothelial carcinoma.

Embodiment 40. The method of either of Embodiments 37 and 38, wherein the cancer is head and neck cancer.

Embodiment 41. The method of either of Embodiments 37 and 38, wherein the cancer is colorectal cancer.

Embodiment 42. The method of either of Embodiments 37 and 38, wherein the cancer is gastric cancer.

Embodiment 43. The method of Embodiment 36, wherein the antibody is an anti-PD-1 monoclonal antibody.

Embodiment 44. The method of Embodiment 43, wherein the anti-PD-1 antibody is chosen from nivolumab, pembrolizumab, cemiplimab, and dostarlimab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies and known in the art.

Embodiment 45. The method of Embodiment 36, wherein the antibody is an anti-CTLA-4 monoclonal antibody.

Embodiment 46. The method of Embodiment 45, wherein the anti-CTLA-4 monoclonal is antibody ipilimumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies and known in the art.

Embodiment 47. The method of Embodiment 37, wherein the anti-PD-L1 monoclonal antibody is chosen from atezolizumab, avelumab, coseibelimab, and durvalumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies disclosed herein or known in the art.

Embodiment 48. The method of Embodiment 47, wherein the anti-PD-L1 monoclonal antibody is avelumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibody disclosed herein or known in the art.

Embodiment 49. The method of Embodiment 1, wherein the bispecific molecule is an NK cell engager or a trispecific killer cell engager.

Embodiment 50. The method of any preceding Embodiment, wherein the memory NK cells are administered before the antibody (or other bispecific molecule).

Embodiment 51. The method of any preceding Embodiment, wherein the memory NK cells are administered concurrently with the antibody (or other bispecific molecule).

Embodiment 52. The method of any preceding Embodiment, wherein the memory NK cells are administered after the antibody (or other bispecific molecule).

Embodiment 53. An immunotherapeutic composition comprising memory NK cells and a bispecific molecule (e.g., a monoclonal antibody, NK cell engager, or trispecific killer cell engager) comprising a tumor antigen binding moiety and a CD16 binding moiety, and a therapeutically acceptable carrier.

Embodiment 54. An immunotherapeutic kit comprising memory NK cells and a bispecific molecule (e.g., a monoclonal antibody, NK cell engager, or trispecific killer cell engager) comprising a tumor antigen binding moiety and a CD16 binding moiety, and optionally, instructions to administer the memory NK cells and antibody in simultaneous or sequential combination to a subject with cancer.

Embodiment 55. The composition or kit of either of Embodiments 53 and 54, wherein the bispecific molecule is a monoclonal antibody of the IgG type.

Embodiment 56. The composition or kit of Embodiment 55, wherein the monoclonal antibody is an IgG1 or an IgG3.

Embodiment 57. The composition or kit of Embodiment 56, wherein the Fc moiety of the IgG1 monoclonal antibody is mutated to enhance CD16 binding affinity.

Embodiment 58. The composition or kit of either of Embodiments 55 and 56, wherein the monoclonal antibody is chosen from an anti-HER2 monoclonal antibody, an anti-PDL-1 monoclonal antibody, and an anti-EGFR monoclonal antibody.

Embodiment 59. The composition or kit of Embodiment 58, wherein the IgG1 (or IgG3) antibody is an anti-HER2 monoclonal antibody.

Embodiment 60. The composition or kit of Embodiment 59, wherein the cancer is ovarian cancer.

Embodiment 61. The composition or kit of Embodiment 60, wherein the ovarian cancer is a clear cell carcinoma.

Embodiment 62. The composition or kit of Embodiment 60, wherein the ovarian cancer is an endometrioid adenocarcinoma.

Embodiment 63. The composition or kit of Embodiment 60, wherein the ovarian cancer is a mucinous adenocarcinoma.

Embodiment 64. The composition or kit of Embodiment 60, wherein the ovarian cancer is a serous carcinoma.

Embodiment 65. The composition or kit of Embodiment 60, wherein the ovarian cancer is a stromal tumor.

Embodiment 66. The composition or kit of Embodiment 60, wherein the ovarian cancer is a germ cell tumor.

Embodiment 67. The composition or kit of Embodiment 60, wherein the ovarian cancer is a small cell cancer of the ovary.

Embodiment 68. The composition or kit of Embodiment 59, wherein the cancer is gastric cancer.

Embodiment 69. The composition or kit of any of Embodiments 59-68, wherein the anti-HER2 monoclonal antibody is chosen from trastuzumab and margetuximab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies disclosed herein or known in the art.

Embodiment 70. The composition or kit of Embodiment 69, wherein the anti-HER2 monoclonal antibody is trastuzumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with the antibody disclosed herein or known in the art.

Embodiment 71. The composition or kit of Embodiment 58, wherein the IgG1 (or IgG3) antibody is an anti-EGFR monoclonal antibody.

Embodiment 72. The composition or kit of Embodiment 71, wherein the cancer is head and neck cancer.

Embodiment 73. The composition or kit of Embodiment 72, wherein the head and neck cancer is oropharyngeal cancer.

Embodiment 74. The composition or kit of Embodiment 72, wherein the head and neck cancer is hypopharyngeal cancer.

Embodiment 75. The composition or kit of Embodiment 72, wherein the head and neck cancer is laryngeal cancer.

Embodiment 76. The composition or kit of Embodiment 72, wherein the head and neck cancer is lip and oral cavity cancer.

Embodiment 77. The composition or kit of Embodiment 72, wherein the head and neck cancer is nasopharyngeal cancer.

Embodiment 78. The composition or kit of Embodiment 72, wherein the head and neck cancer is paranasal sinus cancer.

Embodiment 79. The composition or kit of Embodiment 72, wherein the head and neck cancer is salivary gland cancer.

Embodiment 80. The composition or kit of Embodiment 72, wherein the head and neck cancer is squamous cell neck cancer.

Embodiment 81. The composition or kit of Embodiment 72, wherein the head and neck cancer is nasal cavity cancer.

Embodiment 82. The composition or kit of Embodiment 72, wherein the head and neck cancer is soft tissue sarcoma.

Embodiment 83. The composition or kit of Embodiment 72, wherein the head and neck cancer is thyroid cancer.

Embodiment 84. The composition or kit of Embodiment 71, wherein the cancer is colorectal cancer.

Embodiment 85. The composition or kit of any of Embodiments 71-84, wherein the anti-EGFR monoclonal antibody is chosen from cetuximab, panitumumab, and necitumumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies disclosed herein or known in the art.

Embodiment 86. The composition or kit of Embodiment 85, wherein the anti-EGFR monoclonal antibody is cetuximab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with the antibody disclosed herein or known in the art.

Embodiment 87. The composition or kit of Embodiment 58, wherein the IgG1 (or IgG3) antibody is an anti-immune-checkpoint protein antibody.

Embodiment 88. The composition or kit of Embodiment 87, wherein the anti-immune-checkpoint protein antibody is chosen from an anti-PD-1 monoclonal antibody, an anti-PD-L1 monoclonal antibody, and an anti-CTLA-4 monoclonal antibody.

Embodiment 89. The composition or kit of Embodiment 88, wherein the cancer is chosen from urothelial carcinoma, head and neck cancer, colorectal cancer, and gastric cancer.

Embodiment 90. The composition or kit of Embodiment 89, wherein the antibody is an anti-PD-L1 monoclonal antibody.

Embodiment 91. The composition or kit of Embodiment 90, wherein the anti-PD-L1 monoclonal antibody is avelumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with the antibody disclosed herein or known in the art.

Embodiment 92. The composition or kit of either of Embodiments 90 and 91, wherein the cancer is urothelial carcinoma.

Embodiment 93. The composition or kit of either of Embodiments 90 and 91, wherein the cancer is head and neck cancer.

Embodiment 94. The composition or kit of either of Embodiments 90 and 91, wherein the cancer is colorectal cancer.

Embodiment 95. The composition or kit of either of Embodiments 90 and 91, wherein the cancer is gastric cancer.

Embodiment 96. The composition or kit of Embodiment 89, wherein the antibody is an anti-PD-1 monoclonal antibody.

Embodiment 97. The composition or kit of Embodiment 96, wherein the anti-PD-1 antibody is chosen from nivolumab, pembrolizumab, cemiplimab, and dostarlimab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies and known in the art.

Embodiment 98. The composition or kit of Embodiment 89, wherein the antibody is an anti-CTLA-4 monoclonal antibody.

Embodiment 99. The composition or kit of Embodiment 98, wherein the anti-CTLA-4 monoclonal is antibody ipilimumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with the antibody and known in the art.

Embodiment 100. The composition or kit of Embodiment 90, wherein the anti-PD-L1 monoclonal antibody is chosen from atezolizumab, avelumab, coseibelimab, and durvalumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with those antibodies disclosed herein or known in the art.

Embodiment 101. The composition or kit of Embodiment 100, wherein the anti-PD-L1 monoclonal antibody is avelumab; and optionally, wherein the antibody has at least 90% sequence identity (e.g., at least 91%, at least 92%, at least 93%, at least 94%, at least 95%, at least 96%, at least 97%, at least 98%, or at least 99% sequence identity) to, or the sequences of, the SEQ ID NO.s associated with the antibody disclosed herein or known in the art.

Embodiment 102. The method, composition, or kit of any of the preceding Embodiments, wherein the memory NK cells are administered before the antibody.

Embodiment 103. The method, composition, or kit of any of the preceding Embodiments, wherein the memory NK cells are administered concurrently with the antibody.

Embodiment 104. The method, composition, or kit of any of the preceding Embodiments, wherein the memory NK cells are administered after the antibody.

Embodiment 105. The method, composition or kit of any of the preceding Embodiment, wherein the memory NK cells have increased expression of CD16 relative to normal NK cells.

Embodiment 106. The method, composition, or kit of Embodiment 106 wherein the memory NK cells' increased expression of CD16 relative to normal NK cells persists for at least 30 days.

Embodiments disclosed and not presently claimed herein are not intended to be dedicated to the public.

### Methods of Expansion and Priming of Immune Effector Cells

Expansion of the NK cells in vitro may be performed in an enrichment process that uses an expanding agent comprising cytokines, or, preferably, expansion fusion proteins comprising functional fragments of cytokines, and multichain complexes thereof. For example, the expanding agent may comprise one or more of IL,-2, IL,-4, IL,-7, IL,-9, TL,-15, and IL,-21, or a combination thereof, for example a cocktail of IL,-7, TL,-21, and IL-15, in an amount sufficient to produce a desired quantity or fold expansion of NK cells. Such cytokines may be obtained commercially or made by methods known in the art. Or, for example, the expanding agent may comprise one or more expansion fusion proteins, e.g., may be chosen from amongst multi-chain fusion protein complexes disclosed in WO2020047299, WO202047473, or WO 2020257639, for example 7t15-21s, in an amount sufficient to expand NIK cells. The sequences of 7t15-21s are disclosed in Table 1.

TABLE 1
Sequences of Exemplary Expansion Fusion Protein Chains

| | WO'299 | | |
|---|---|---|---|
| | SEQ ID | | |
| Element | NO. | SEQ ID NO. | Sequence |
| Mature 7t15 | 104 | SEQ ID NO: 1 | DCDIEGKDGKQYESVLMVSIDQLLDSMKEI |
| | | | GSNCLNNEFNFFKRHICDANKEGMFLFRAA |
| | | | RKLRQFLKMNSTGDFDLHLLKVSEGTTILLN |
| | | | CTGQVKGRKPAALGEAQPTKSLEENKSLKE |
| | | | QKKLNDLCFLKRLLQEIKTCWNKILMGTKE |
| | | | HSGTTNTVAAYNLTWKSTNFKTILEWEPKP |
| | | | VNQVYTVQISTKSGDWKSKCFYTTDTECDL |
| | | | TDEIVKDVKQTYLARVFSYPAGNVESTGSA |
| | | | GEPLYENSPEFTPYLETNLGQPTIQSFEQVGT |
| | | | KVNVTVEDERTLVRRNNTFLSLRDVFGKDL |
| | | | IYTLYYWKSSSSGKKTAKTNTNEFLIDVDK |
| | | | GENYCFSVQAVIPSRTVNRKSTDSPVECMG |
| | | | QEKGEFRENWVNVISDLKKIEDLIQSMHIDA |
| | | | TLYTESDVHPSCKVTAMKCFLLELQVISLES |
| | | | GDASIHDTVENLIILANNSLSSNGNVTESGC |
| | | | KECEELEEKNIKEFLQSFVHIVQMFINTS |
| Mature 21s | 108 | SEQ ID NO: 2 | QGQDRHMIRMRQLIDIVDQLKNYVNDLVPE |
| | | | FLPAPEDVETNCEWSAFSCFQKAQLKSANT |
| | | | GNNERIINVSIKKLKRKPPSTNAGRRQKHRL |
| | | | TCPSCDSYEKKPPKEFLERFKSLLQKMIHQH |
| | | | LSSRTHGSEDSITCPPPMSVEHADIWVKSYS |
| | | | LYSRERYICNSGFKRKAGTSSLTECVLNKAT |
| | | | NVAHWTTPSLKCIR |

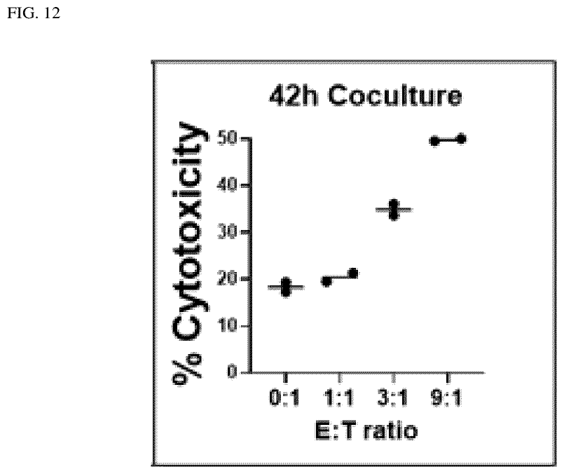

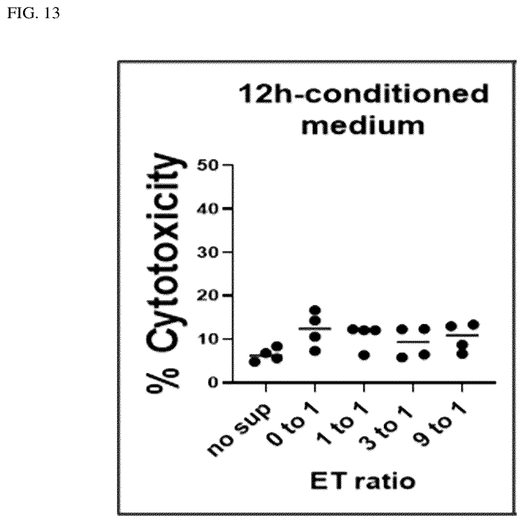

Expansion is additionally facilitated by use of a cross-linking agent, such as an antibody targeting a linking domain of the fusion proteins disclosed above, for example an anti-tissue-factor antibody. Examples of anti-tissue factor antibodies are known in the art. WO202047473 and WO2020257639 disclose the a-TF Ab to be used. See also, US 8,007,795 and WO2003037911, in particular IgG1 humanized antibodies incorporating the CDRs of the H36 hybridoma and humanized framework regions LC-08 (FIG. 12 of that application) and HC-09 (FIG. 13 of that application). Table 2 below discloses the sequences of the a-TF Ab believed to be used in WO′473 and WO′639, disclosed in US′795 and WO′911, obtained from HCW Biologics, and used in the experiments below unless otherwise stated, referred to herein as ATF1. ATF1 HCDR2 is one of the two sequences below. Accordingly, an expansion agent as disclosed herein may comprise a combination of one or more cytokines or an EFP as disclosed above, together with a crosslinking agent such as ATF11, the sequence(s) of which are disclosed in Table 2. Additional anti-TF antibodies are known in the art, and can be readily obtained from commercial sources.

TABLE 2
Sequences of Exemplary Anti-TF Antibodies (“ATF1”)

| a-TF | US'795 | | |
|---|---|---|---|
| Antibody | SEQ ID | SEQ ID | |
| Element | NO. | NO. | Sequence |
| LC-CDR1 | 116 | SEQ ID | LASQTIDTWLA |
| | | NO: 3 | |
| LC-CDR2 | 6 | SEQ ID | AATNLAD |
| | | NO: 4 | |
| LC-CDR3 | 7 | SEQ ID | QQVYSSPFT |
| | | NO: 5 | |
| LC-FR1 | 109 | SEQ ID | DIQMTQSPASLSASVGDRVTITC |
| | | NO: 6 | |
| LC-FR2 | 108 | SEQ ID | WYLQKPGKSPQLLIY |
| | | NO: 7 | |
| LC-FR3 | 112 | SEQ ID | GVPSRFSGSGSGTDFSFTISSLQPEDFATY |
| | | NO: 8 | YC |
| LC-FR4 | 110 | SEQ ID | FGQGTKLEIK |
| | | NO: 9 | |
| VL | n/a | SEQ ID | DIQMTQSPASLSASVGDRVTITCLASQTID |
| | | NO: 10 | TWLAWYLQKPGKSPQLLIYAATNLADG |
| | | | VPSRFSGSGSGTDFSFTISSLQPEDFATYY |
| | | | CQQVYSSPFT GQGTKLEIK |
| HC-CDR1 | 134 | SEQ ID | DYNVY |
| | | NO: 11 | |
| HC-CDR2 (a) | 9 | SEQ ID | YIDPYNGITIYDQNFKG |
| | | NO: 12 | |
| HC-CDR2 (b) | 101 | SEQ ID | YIDPYNGITIYDQNLKG |
| | | NO: 13 | |
| HC-CDR3 | 10 | SEQ ID | DVTTALDF |
| | | NO: 14 | |
| HC-FR1 | 129 | SEQ ID | QIQLVQSGGEVKKPGASVRVSCKASGYS |
| | | NO: 15 | FT |
| HC-FR2 | 123 | SEQ ID | WVRQSPGKGLEWIG |
| | | NO: 16 | |
| HC-FR3 | 126 | SEQ ID | KATLTVDKSTSTAYMELSSLRSEDTAVYF |
| | | NO: 17 | CAR |
| HC-FR4 | 122 | SEQ ID | WGQGTTVTVSS |
| | | NO: 18 | |
| VH-1 | n/a | SEQ ID | QIQLVQSGGEVKKPGASVRVSCKASGYS |
| | | NO: 19 | FTDYNVYWVRQSPGKGLEWIGYIDPYNG |
| | | | ITIYDQNFKGKATLTVDKSTSTAYMELSS |
| | | | LRSEDTAVYFCARDVTTALDFWGQGTTV |
| | | | TVSS |
| VH-2 | n/a | SEQ ID | QIQLVQSGGEVKKPGASVRVSCKASGYS |
| | | NO: 20 | FTDYNVYWVRQSPGKGLEWIGYIDPYNG |
| | | | ITIYDQNLKGKATLTVDKSTSTAYMELSS |
| | | | LRSEDTAVYFCARDVTTALDFWGQGTTV |
| | | | TVSS |
| IgG1 LC | 97 | SEQ ID | RTVAAPSVFIFPPSDEQLKSGTASVVCLL |
| constant | | NO: 21 | NNFYPREAKVQWKVDNALQSGNSQESV |
| region | | | TEQDSKDSTYSLSSTLTLSKADYEKHKVY |
| | | | ACEVTHQGLSSPVTKSFNRGEC |
| IgG1 HC | 98 | SEQ ID | RTVAAPSVFIFPPSDEQLKSGTASVVCLL |
| constant | | NO: 22 | NNFYPREAKVQWKVDNALQEFASTKGPS |
| region | | | VFPLAPSSKSTSGGTAALGCLVKDYFPEP |
| | | | VTVSWNSGALTSGVHTFPAVLQSSGLYS |
| | | | LSSVVTVPSSSLGTQTYICNVNHKPSNTK |
| | | | VDKKVEPKSCDKTHTCPPCPAPELLGGPS |
| | | | VFLFPPKPKDTLMISRTPEVTCVVVDVSH |
| | | | EDPEVKFNWYVDGVEVHNAKTKPREEQ |
| | | | YNSTYRVVSVLTVLHQDWLNGKEYKCK |
| | | | VSNKALPAPIEKTISKAKGQPREPQVYTL |
| | | | PPSRDELTKNQVSLTCLVKG+C7FYPSDIA |
| | | | VEWESNGQPENNYKTTPPVLDSDGSFFL |
| | | | YSKLTVDKSRWQQGNVFSCSVMHEALH |
| | | | NHYTQKSLSLSPGK |

Alternative methods of cross-linking are known in the art, and include functionalized microparticles (beads), feeder cells and plasma membrane particles. Feeder-free systems are often preferred. For example, R&D Systems Cloudz human NIK cell expansion kits, employing dissolvable sodium alginate microspheres that are functionalized with anti-CD2 and anti-NKp46 antibodies, may be used with expansion cytokines (or fragments thereof, or fusion proteins comprising) and combinations thereof as disclosed herein, along with a release buffer after expansion for quickly dissolving microparticles, facilitating cell harvesting.

Priming to obtain the memory like character is performed with a priming agent comprising a combination of stimulatory cytokines, such as one or more of IL,-12, IL,-23, IL-27, and IL-35; one or more of IL-2, IL-4, IL-7, IL-9, IL-15, and IL-21; and one or more of IL-18, IL-1a, IL-1b, IL-36a, IL-36b, and IL-36g. Alternatively, the priming agent may comprise priming fusion proteins comprising functional fragments of cytokines, and multichain complexes thereof. For example, the fusion proteins may be chosen from amongst multi-chain fusion protein complexes disclosed in WO2020047299, WO202047473, or WO 2020257639, for example 18t15-12s (HCW-9201), the sequences of which are disclosed in Table 3.

TABLE 3
Sequences of Exemplary Priming Fusion Protein Chains

| | WO'299 | | |
|---|---|---|---|
| | SEQ ID | SEQ ID | |
| Element | NO. | NO. | Sequence |
| Mature 18t15 | 70 | SEQ ID | YFGKLESKLSVIRNLNMTDSDCRDNAPRTI |
| | | NO: 23 | FIISMYKDSQPRGMAVTISVKCEKISTLSCE |
| | | | NKIISFKEMNPPDNIKDTKSDIIFFQRSVPGH |
| | | | DNKMQFESSSYEGYFLACEKERDLFKLILK |
| | | | KEDELGDRSIMFTVQNEDSGTTNTVAAYN |
| | | | LTWKSTNFKTILEWEPKPVNQVYTVQISTK |
| | | | SGDWKSKCFYTTDTECDLTDEIVKDVKQT |
| | | | YLARVFSYPAGNVESTGSAGEPLYENSPEF |
| | | | TPYLETNLGQPTIQSFEQVGTKVNVTVEDE |
| | | | RTLVRRNNTFLSLRDVFGKDLIYTLYYWKS |
| | | | SSSGKKTAKTNTNEFLIDVDKGENYCFSVQ |
| | | | AVIPSRTVNRKSTDSPVECMGQEKGEFREN |
| | | | WVNVISDLKKIEDLIQSMHIDATLYTESDV |
| | | | HPSCKVTAMKCFLLELQVISLESGDASIHD |
| | | | TVENLIILANNSLSSNGNVTESGCKECEELE |
| | | | EKNIKEFLQSFVHIVQMFINTS |
| Mature 12s | 74 | SEQ ID | IWELKKDVYVVELDWYPDAPGEMVVLTC |
| | | NO: 24 | DTPEEDGITWTLDQSSEVLGSGKTLTIQVK |
| | | | EFGDAGQYTCHKGGEVLSHSLLLLHKKED |
| | | | GIWSTDILKDQKEPKNKTFLRCEAKNYSGR |
| | | | FTCWWLTTISTDLTFSVKSSRGSSDPQGVT |
| | | | CGAATLSAERVRGDNKEYEYSVECQEDSA |
| | | | CPAAEESLPIEVMVDAVHKLKYENYTSSFF |
| | | | IRDIIKPDPPKNLQLKPLKNSRQVEVSWEYP |
| | | | DTWSTPHSYFSLTFCVQVQGKSKREKKDR |
| | | | VFTDKTSATVICRKNASISVRAQDRYYSSS |
| | | | WSEWASVPCSGGGGSGGGGSGGGGSRNL |
| | | | PVATPDPGMFPCLHHSQNLLRAVSNMLQK |
| | | | ARQTLEFYPCTSEEIDHEDITKDKTSTVEAC |
| | | | LPLELTKNESCLNSRETSFITNGSCLASRKT |
| | | | SFMMALCLSSIYEDLKMYQVEFKTMNAKL |
| | | | LMDPKRQIFLDQNMLAVIDELMQALNENS |
| | | | ETVPQKSSLEEPDFYKTKIKLCILLHAFRIR |
| | | | AVTIDRVMSYLNASITCPPPMSVEHADIWV |
| | | | KSYSLYSRERYICNSGFKRKAGTSSLTECV |
| | | | LNKATNVAHWTTPSLKCIR |

Accordingly, provided herein are memory natural killer (NK) cells produced by, sequentially:
a) purifying a population of NK cells;
b) priming the NK cells; and
c) expanding the NK cells.

Also provided herein are memory natural killer (NK) cells produced by, sequentially:
a) purifying a population of NK cells;
b) expanding the NK cells; and
c) priming the NK cells.

Also provided herein are memory natural killer (NK) cells produced by:
a) purifying a population of NK cells; and
b) concurrently priming and expanding the NK cells.

In some embodiments, the NK cell population is purified starting from donor blood, or fresh or previously cryopreserved leukapheresate. In some embodiments, the purification is performed via positive selection (for example on the Miltenyi CliniMACS Prodigy). In some embodiments, the purification is performed via negative selection (for example, the StemCell EasySep NK Cell Enrichment Kit). In some embodiments, purification is performed using a combination of positive and negative selection. In some embodiments, the NK cells are differentiated from lymphoid progenitor cells.

In some embodiments, the NK cells are expanded by exposure to an expansion agent comprising a combination of cytokines, or functional fragments thereof, and/or fusion proteins comprising functional fragments thereof, or a combination of any of the foregoing, and optionally a crosslinking agent.

In some embodiments, the NK cells are expanded by exposure to an expansion agent comprising:
one or more of IL-2, IL-4, IL-7, IL-9, IL-15, and IL-21, or functional fragments thereof; or
fusion proteins comprising functional fragments one or more of IL-2, IL-4, IL-7, IL-9, IL-15, and IL-21;
and optionally a crosslinking agent; or
microspheres functionalized with NK-cell crosslinking antibodies and expansion cytokines;
   or a combination of any of the foregoing.

In some embodiments, the NK cells are expanded by exposure to an expansion agent comprising a combination of IL-7, IL-21, and IL-15, or functional fragments thereof, and/or fusion proteins comprising functional fragments thereof, or a combination of any of the foregoing.

In some embodiments, the NK cells are expanded by exposure to an expansion agent comprising fusion proteins comprising functional fragments of IL-7, IL-21, and IL-15.

In some embodiments, the NK cells are expanded by exposure to an expansion agent comprising 7t15-21s.

In some embodiments, the expansion agent comprises a crosslinking agent. In some embodiments, the crosslinking agent is a crosslinking antibody. In some embodiments, the crosslinking antibody is ATF1.

In some embodiments, the NK cells are expanded by exposure to an expansion agent comprising 7t15-21s and ATF1.

In some embodiments, the NK cells are expanded by exposure to an expansion agent for 1 day to 40 days. In some embodiments, the NK cells are expanded by exposure to an expansion agent for 7 days to 21 days. In some embodiments, the NK cells are expanded by exposure to an expansion agent for about 14 days.

In some embodiments, the expansion agent comprises 7t15-21s and ATF1. In some embodiments, the expansion agent comprises 7t15-21s at a concentration of 0.1-300 nM and ATF1 at a concentration of 0.01-200 nM. In some embodiments, the expansion agent comprises 7t15-21s at a concentration of 0.2-200 nm and ATF1 at a concentration of 0.01-100 nM. In some embodiments, the expansion agent comprises 7t15-21s at a concentration of about 50 nM and ATF1 at a concentration of about 25 nM.

In some embodiments, the NK cells are expanded by exposure to 7t15-21s and ATF1 for about 14 days. In some embodiments, the NK cells are expanded by exposure to 7t15-21s at a concentration of about 50 nM and ATF1 at a concentration of about 25 nM for about 14 days.

In some embodiments, the NK cells are primed by exposure to a priming agent, for example chosen from a combination of cytokines, or functional fragments thereof, and/or fusion proteins comprising functional fragments thereof, or a combination of any of the foregoing.

In some embodiments, the NK cells are expanded to greater than 10 times the starting number. In some embodiments, the NK cells are expanded to greater than 100 times the starting number. In some embodiments, the NK cells are expanded to greater than 1000 times the starting number.

In some embodiments, the NK cells are primed by exposure to a priming agent comprising:
one or more of IL-12, IL-23, IL-27, and IL-35;
one or more of IL-2, IL-4, IL-7, IL-9, IL-15, and IL-21; and
one or more of IL-18, IL-1a, IL-1b, IL-36a, IL-36b, and IL-36g;
   or functional fragments thereof, and/or fusion proteins comprising functional fragments thereof, or a combination of any of the foregoing.

In some embodiments, the NK cells are primed by exposure to a priming agent comprising a combination of IL-12, IL-15, and IL-18.

In some embodiments, the NK cells are primed by exposure to a priming agent comprising fusion proteins comprising functional fragments of IL-12, IL-15, and IL-18. In some embodiments, the NK cells are primed by exposure to a priming agent comprising fusion protein 18t15-12s.

In some embodiments, the NK cells are primed with 18t15-12s at a concentration of 200-300 nM. In some embodiments, the NK cells are primed with 18t15-12s at a concentration of 250 nM.

In some embodiments, the NK cells are primed for 1 minute to 24 hours. In some embodiments, the NK cells are primed for 0.5 to 16 hours. In some embodiments, the NK cells are primed for 1 to 3 hours.

In some embodiments, the NK cells are cryopreserved.

In some embodiments, the memory-like phenotype is indicated by the level of expression of cell-surface CD69, CD25, CD16, and/or NKG2A.

In some embodiments, the memory NK cells have one or more of:
a) improved cytotoxicity against cancer cells;
b) improved persistence;
c) improved anti-tumor activity; and/or
d) increased production of cytokines;
compared to NK cells which have not been primed.

In some embodiments, the produced cytokines are chosen from IFNg, TNFa, GM-CSF, and combinations thereof.

In any of the Enumerated Embodiments above, the memory NK cells may be further limited by these foregoing embodiments.

### Immune Effector Cells

Immune effector cells as disclosed herein include NK cells and subtypes thereof, such as memory NK cells, memory-like (ML) NK cells, and cytokine-induced memory-like (CIML) NK cells, and variations thereof, any of which may be derived from various sources, including peripheral or cord blood cells, stem cells, induced pluripotent stem cells (iPSCs), and immortalized NK cells such as NK-92 cells.

### NK Cells

Natural killer (NK) cells are traditionally considered innate immune effector lymphocytes which mediate host defense against pathogens and antitumor immune responses by targeting and eliminating abnormal or stressed cells not by antigen recognition or prior sensitization, but through the integration of signals from activating and inhibitory receptors. Natural killer (NK) cells are an alternative to T cells for allogeneic cellular immunotherapy since they have been administered safely without major toxicity, do not cause graft versus host disease (GvHD), naturally recognize and eliminate malignant cells, and are amendable to cellular engineering.

### Memory, Memory-Like, and CIML NK Cells

In addition to their innate cytotoxic and immunostimulatory activity, NK cells constitute a heterogeneous and versatile cell subset, including persistent memory NK populations, in some cases also called memory-like or cytokine-induced-memory-like (CIMIL) NK cells, that mount robust recall responses. Memory NK cells can be produced by stimulation by pro-inflammatory cytokines or activating receptor pathways, either naturally or artificially (“priming”). Memory NK cells produced by cytokine activation have been used clinically in the setting of leukemia immunotherapy.

Increased CD56, Ki-67, NKG2A, and increased activating receptors NKG2D, NKp30, and NKp44 have been observed in in vivo differentiated memory NK cells. In addition, in vivo differentiation showed modest decreases in the median expression of CD16 and CD11b. Increased frequency of TRAIL, CD69, CD62L, NKG2A, and NKp30-positive NK cells were observed in ML NK cells compared with both ACT and BL NK cells, whereas the frequencies of CD27+ and CD127+ NK cells were reduced. Finally, unlike in vitro differentiated ML NK cells, in vivo differentiated ML NK cells did not express CD25.

### Cytokine-Induced Memory-Like Natural Killer Cells (CIML-NKs)

NK cells may be induced to acquire a memory-like phenotype, for example by priming (preactivation) with combinations of cytokines, such as interleukin-12 (IL-12), IL-15, and IL-18. These cytokine-induced memory-like (CIML) NK cells (CIML-NKs or CIMLs) exhibit enhanced response upon restimulation with the cytokines or triggering via activating receptors. CIML NK cells may be produced by activation with cytokines such as IL-12, IL-15, and IL-18 and/or their related family members, or functional fragments thereof, or fusion proteins comprising functional fragments thereof.

Memory NK cells typically exhibit differential cell surface protein expression patterns when compared to traditional NK cells. Such expression patterns are known in the art and may comprise, for example, increased CD56, CD56 subset CD56dim, CD56 subset CD56bright, CD16, CD94, NKG2A, NKG2D, CD62L, CD25, NKp30, NKp44, and NKp46 (compared to control NK cells) in CIML NK cells (see e.g., Romee et al. Sci Transl Med. 2016 Sep. 21; 8(357):357). Memory NK cells may also be identified by observed in vitro and in vivo properties, such as enhanced effector functions such as cytotoxicity, improved persistence, increased IFN-7 production, and the like, when compared to a heterogenous NK cell population.

### Antibodies

Provided herein are antibodies comprising the polypeptides disclosed herein. In some embodiments the antibodies comprise the light and heavy chains, the VH and VL chains, or the groupings of CDRs disclosed herein and/or known on the art.

Various forms of antibodies disclosed are contemplated herein. For example, the antibodies can have human frameworks and constant regions of the isotypes, IgA, IgD, IgE, IgG, and IgM, more particularly, IgG1, IgG2, IgG3, IgG4, and in some cases with various mutations to alter Fc receptor function or prevent Fab arm exchange or an antibody fragment, e.g., a F(ab′)2 fragment, a F(ab) fragment, a single chain Fv fragment (scFv), etc.

In certain embodiments, an antibody provided herein is a human antibody. Human antibodies can be produced using various techniques known in the art. For example, human antibodies may also be generated by isolating Fv clone variable domain sequences chosen from human-derived phage display libraries. Such variable domain sequences may then be combined with a desired human constant domain.

An antibody as provided herein may be a chimeric antibody, e.g. comprising a non-human variable region (e.g., a variable region derived from a mouse, rat, hamster, rabbit, or non-human primate, such as a monkey) and a human constant region, or a “class switched” antibody in which the class or subclass has been changed from that of the parent antibody.

An antibody as provided herein may be a humanized antibody. Typically, a non-human antibody is humanized to reduce immunogenicity to humans, while retaining the specificity and affinity of the parental non-human antibody. Generally, a humanized antibody comprises one or more variable domains in which HVRs, e.g., CDRs, (or portions thereof) are derived from a non-human antibody, and FRs (or portions thereof) are derived from human antibody sequences. A humanized antibody optionally will also comprise at least a portion of a human constant region. In some embodiments, some FR residues in a humanized antibody are substituted with corresponding residues from a non-human antibody (e.g., the antibody from which the HVR residues are derived), e.g., to restore or improve antibody specificity or affinity.

Antibodies disclosed herein may also be bispecific or trispecific—i.e., that comprise an antigen-recognition domain that comprises one of the polypeptides disclosed herein and one or more other antigen-recognition domains that binds to another antigen. For example, one arm of the antibody may bind a polymorph of an antigen on a cancer cell, and the other arm may bind EGFR, HER-2, or PD-L1, or another cancer cell target. In an example of a trispecific antibody, the antibody would also bind another target such as CD16 to enhance activity of recruited memory NK cells.

In some embodiments, a humanized antibody comprises, in addition to the variable regions, a human acceptor framework, e.g. a human immunoglobulin framework or a human consensus framework. Human framework regions that may be used for humanization include but are not limited to framework regions selected using the “best-fit” method, framework regions derived from the consensus sequence of human antibodies of a particular subgroup of light or heavy chain variable regions, human mature (somatically mutated) framework regions or human germline framework regions, and framework regions derived from screening FR libraries.

In certain embodiments, an antibody provided herein is a multispecific antibody, e.g. a bispecific antibody. Multispecific antibodies are monoclonal antibodies that have binding specificities for at least two different sites. For example, one of the binding specificities is for EGFR, HER-2, or PD-L1, and the other is for any other antigen. In certain embodiments, bispecific antibodies may bind to two different epitopes of the same antigen. Bispecific antibodies may also be used to localize cytotoxic agents to cells which express a target antigen. Bispecific antibodies can be prepared as full length antibodies or antibody fragments. Techniques for making multispecific antibodies include, but are not limited to, recombinant co-expression of two immunoglobulin heavy chain-light chain pairs having different specificities, “knob-in-hole” engineering, engineering electrostatic steering effects for making antibody Fc-heterodimeric molecules, cross-linking two or more antibodies or fragments, using leucine zippers to produce bi-specific antibodies, using “diabody” technology for making bispecific antibody fragments, and using single-chain Fv (scFv) dimers.

Amino acid sequence variants of the antibodies provided herein are contemplated. For example, it may be desirable to improve the binding affinity and/or other biological properties of the antibody. Amino acid sequence variants of an antibody may be prepared by introducing appropriate modifications into the nucleotide sequence encoding the antibody, or by peptide synthesis. Such modifications include, for example, deletions from, and/or insertions into and/or substitutions of residues within the amino acid sequences of the antibody. Any combination of deletion, insertion, and substitution can be made to arrive at the final construct, provided that the final construct possesses the desired characteristics, e.g., antigen-binding.

Sites of interest for substitutional mutagenesis include the variable regions and framework regions. Amino acids may be grouped according to common side-chain properties:
(1) hydrophobic: Norleucine, Met, Ala, Val, Leu, He;
(2) neutral hydrophilic: Cys, Ser, Thr, Asn, Gin;
(3) acidic: Asp, Glu;
(4) basic: His, Lys, Arg;
(5) residues that influence chain orientation: Gly, Pro;
(6) aromatic: Trp, Tyr, Phe.

Amino acid substitutions may be introduced into an antibody of interest and the products screened for a desired activity, e.g., retained/improved antigen binding, decreased immunogenicity, improved ADCC or CDC, and/or altered pharmacokinetic properties such as extended half-life. Conservative substitutions are known in the art. Examples of such substitutions include the LS and YTE mutations to the Fc region. Non-conservative substitutions will entail exchanging a member of one of these classes for another class.

Antibodies may also comprise modifications to glycan chains substituting certain residues such as Asn 297. For example, antibodies may be engineered or treated to be afucosylated to improve ADCC.

Antibodies and other proteins and peptides disclosed herein may comprise amino acid sequences varying from their sequences disclosed herein or known in the art, e.g., having at least 90% sequence identity (e.g., at least 91%, at least 92%, at least 93%, at least 94%, at least 95%, at least 96%, at least 97%, at least 98%, or at least 99% sequence identity) to, or the sequences of, the sequences disclosed herein or known in the art. In some embodiments, the heavy chain, the light chain, the VH domain, the VL domain, and/or one or more of the CDRs has at least 95%, at least 97%, at least 98% or at least 99% sequence identity to one of the recited amino acid sequences.

Antibodies comprising the CDRs, variable heavy and light chains disclosed herein may be made by methods known in the art. For example, variable antibody domains may be cloned into IgG expression vectors (IgG conversion). PCR-amplified DNA fragments of heavy and light chain V-domains may be inserted in frame into, e.g., a human IgG1 constant heavy chain containing recipient mammalian expression vector. Antibody expression may be driven by an MPSV promoter and transcription terminated by a synthetic polyA signal sequence located downstream of the CDS.

Antibodies may be produced using recombinant methods and compositions. Nucleic acids encoding the antibodies described herein are provided. Such a nucleic acid may encode an amino acid sequence comprising the VL and/or an amino acid sequence comprising the VH of the antibody (e.g., the light and/or heavy chains of the antibody). Expression vectors comprising (i.e., transformed with) such nucleic acids are provided, as are host cells comprising such nucleic acids. In one such embodiment, a host cell comprises (1) a vector comprising a nucleic acid that encodes an amino acid sequence comprising the VL and an amino acid sequence comprising the VH, or (2) a first vector comprising a nucleic acid that encodes an amino acid sequence comprising the VL of the antibody and a second vector comprising a nucleic acid that encodes an amino acid sequence comprising the VH of the antibody.

Suitable host cells for cloning or expression of antibody-encoding vectors include other prokaryotic or eukaryotic cells described herein. For example, antibodies may be produced in bacteria (e.g., E. coli), in particular when glycosylation and Fc effector function are not needed. In addition to prokaryotes, eukaryotic microbes such as filamentous fungi or yeast are suitable cloning or expression hosts for antibody-encoding vectors, including fungi and yeast strains whose glycosylation pathways have been “humanized,” resulting in the production of an antibody with a partially or fully human glycosylation pattern. Additional suitable host cells for the expression of glycosylated antibody are also derived from multicellular organisms (invertebrates and vertebrates). Examples of invertebrate cells include plant and insect cells. Numerous baculoviral strains have been identified which may be used in conjunction with insect cells, particularly for transfection of Spodoptera frugiperda cells. Plant cell cultures can also be utilized as hosts. In some embodiments, the host cell may be eukaryotic, e.g. a Chinese Hamster Ovary (CHO) cell or lymphoid cell (e.g., Y0, NS0, Sp20 cell). Host cells comprising a nucleic acid encoding the antibody may be cultured under conditions suitable for expression, and the antibody recovered from the host cell or culture medium.

In some embodiments, an antibody provided herein has a dissociation constant (Kd) of <IμM, <100 nM, <50 nM, <10 nM, <5 nM, <1 nM, <0.1 nM, <0.01 nM, or <0.001 nM, and optionally is >10⁻³¹ M. (e.g. 10⁻⁸ M or less, e.g. from 10⁻⁸ M to 10⁻¹³ M, e.g; from 10⁻⁹ M to 10⁻¹³ M). In one embodiment, Kd is measured by a radiolabeled antigen binding assay (RIA) performed with the Fab version of an antibody of interest and its antigen, or using a surface plasmon resonance assay, e.g., WO2015089344.

Antibodies to be administered according to the methods, and/or as part of the pharmaceutical, or immunotherapeutic, compositions or kits disclosed herein, include anti-EGFR antibodies, anti-HER2 antibodies, anti-VEGFR2 antibodies, and anti-PD-L1 antibodies, as well as related compounds such as multispecific antibodies that bind one or more of these and, optionally, also an NK cell antigen. Clinical stage examples of each of these antibodies are given below; many more preclinical-stage biologic drug candidate antibodies and related compounds are known in the art and in various stages of development.

Anti-EGFR antibodies include cetuximab, panitumumab, and necitumumab. In some embodiments, the anti-EGFR antibody is chosen from panitumumab necitumumab.

Anti-HER2 antibodies include trastuzumab (and its Fc glycoengineered counterpart, timigutuzumab), margetuximab, and by some definitions, pertuzumab. In some embodiments, the anti-HER-2 antibody is chosen from trastuzumab and margetuximab.

Checkpoint inhibitors are compounds which inhibit immune checkpoints, for example PD-1, PD-L1, and CTLA-4. Anti-PD-1 antibodies include nivolumab, pembrolizumab, cemiplimab, and dostarlimab. Anti-PD-L1 antibodies include atezolizumab, avelumab, and durvalumab. Anti-CTLA-4 antibodies include ipilimumab. In some embodiments, the antibody is chosen from one of these. In some embodiments, the antibody is an anti-PD-L1 antibody, for example avelumab.

Anti-VEGFR2 antibodies include ramucirumab. In some embodiments, the anti-VEGFR2 antibody is ramucirumab.

Provided below are sequences of selected clinical-stage antibodies, known in the art.

### Cetuximab

Cetuximab and its methods of preparation can be found in, e.g., WO2011059762A1, which is hereby incorporated by reference in its entirety.

Heavy Chain CDRs-Cetuximab

| HCDR1 | HCDR2 | HCDR3 |
|---|---|---|
| NYGVH | VIWSGGNTDYNTPFTS | ALTYYDYEFAY |
| (SEQ ID NO: 25) | (SEQ ID NO: 26) | (SEQ ID NO: 27) |

Light Chain CDRs-Cetuximab

| LCDR1 | LCDR2 | LCDR3 |
|---|---|---|
| RASQSIGTNIH | YASESIS | QQNNNWPTT |
| (SEQ ID NO: 8) | (SEQ ID NO: 29) | (SEQ ID NO: 30) |

| | |
|---|---|
| Heavy | QVQLKQSGPGLVQPSQSLSITCTVSGFSLTNYGVHWVRQSPGKGLEWLGVI |
| Chain (HC) | WSGGNTDYNTPFTSRLSINKDNSKSQVFFKMNSLQSNDTAIYYCARALTYY |
| | DYEFAYWGQGTLVTVSAASTKGPSVFPLAPSSKSTSGGTAALGCLVKDYFP |
| | EPVTVSWNSGALTSGVHTFPAVLQSSGLYSLSSVVTVPSSSLGTQTYICNVN |
| | HKPSNTKVDKRVEPKSCDKTHTCPPCPAPELLGGPSVFLFPPKPKDTLMISR |
| | TPEVTCVVVDVSHEDPEVKFNWYVDGVEVHNAKTKPREEQYNSTYRVVS |
| | VLTVLHQDWLNGKEYKCKVSNKALPAPIEKTISKAKGQPREPQVYTLPPSR |
| | EEMTKNQVSLTCLVKGFYPSDIAVEWESNGQPENNYKTTPPVLDSDGSFFL |
| | YSKLTVDKSRWQQGNVFSCSVMHEALHNHYTQKSLSLSPGK |
| | (SEQ ID NO: 31) |
| Light Chain | DILLTQSPVILSVSPGERVSFSCRASQSIGTNIHWYQQRTNGSPRLLIKYASES |
| (LC) | ISGIPSRFSGSGSGTDFTLSINSVESEDIADYYCQQNNNWPTTFGAGTKLELK |
| | RTVAAPSVFIFPPSDEQLKSGTASVVCLLNNFYPREAKVQWKVDNALQSGN |
| | SQESVTEQDSKDSTYSLSSTLTLSKADYEKHKVYACEVTHQGLSSPVTKSFN |
| | RGEC (SEQ ID NO: 32) |

### Panitumumab

Panitumumab and its methods of preparation can be found in, e.g., U.S. Pat. Nos. 6,235,883 and 7,807,798, which are hereby incorporated by reference in their entirety.

Heavy Chain CDRs-Panitumumab

| HCDR1 | HCDR2 | HCDR3 |
|---|---|---|
| SGDYYWT | HIYYSGNTNYNPSLKS | RVTGAFDI |
| (SEQ ID NO: 33) | (SEQ ID NO: 4) | (SEQ ID NO: 35) |

Light Chain CDRs-Panitumumab

| LCDR1 | LCDR2 | LCDR3 |
|---|---|---|
| QASQDISNYLN | DASNLET | QHFDHLPLA |
| (SEQ ID NO: 36) | (SEQ ID NO: 37) | (SEQ ID NO: 38) |

| | |
|---|---|
| Heavy Chain | QVQLQESGPGLVKPSETLSLTCTVSGGSVSSGDYYWTWIRQSPGKGLEWI |
| (HC) | GHIYYSGNTNYNPSLKSRLTISIDTSKTQFSLKLSSVTAADTAIYYCVRDRV |
| | TGAFDIWGQGTMVTVSSASTKGPSVFPLAPCSRSTSESTAALGCLVKDYFP |
| | EPVTVSWNSGALTSGVHTFPAVLQSSGLYSLSSVVTVPSSNFGTQTYTCNV |
| | DHKPSNTKVDKTVERKC (SEQ ID NO: 39) |
| Light Chain | DIQMTQSPSSLSASVGDRVTITCQASQDISNYLNWYQQKPGKAPKLLIYDA |
| (LC) | SNLETGVPSRFSGSGSGTDFTFTISSLQPEDIATYFCQHFDHLPLAFGGGTK |
| | VEIKRTVAAPSVFIFPPSDEQLKSGTASVVCLLNNFYPREAKVQWKVDNAL |
| | QSGNSQESVTEQDSKDSTYSLSSTLTLSKADYEKHKVYACEVTHQGLSSPV |
| | TKSFNRGEC (SEQ ID NO: 40) |

### Margetuximab

Margetuximab and its methods of preparation can be found in, e.g., WO2021133653A1, which is hereby incorporated by reference in its entirety.

Heavy Chain CDRs-Margetuximab

| HCDR1 | HCDR2 | HCDR3 |
|---|---|---|
| DTYIH | RIYPTNGYTRYDPKFQD | WGGDGFYAMDY |
| (SEQ ID NO: 41) | (SEQ ID NO: 42) | (SEQ ID NO: 43) |

Light Chain CDRs-Margetuximab

| LCDR1 | LCDR2 | LCDR3 |
|---|---|---|
| KASQDVNTAVA | SASFRYT | QQHYTTPPT |
| (SEQ ID NO: 44) | (SEQ ID NO: 45) | (SEQ ID NO: 46) |

| | |
|---|---|
| Heavy Chain | QVQLQQSGPELVKPGASLKLSCTASGFNIKDTYIHWVKQRPEQGLEWIGRI |
| (HC) | YPTNGYTRYDPKFQDKATITADTSSNTAYLQVSRLTSEDTAVYYCSRWGG |
| | DGFYAMDYWGQGASVTVSSASTKGPSVFPLAPSSKSTSGGTAALGCLVKD |
| | YFPEPVTVSWNSGALTSGVHTFPAVLQSSGLYSLSSVVTVPSSSLGTQTYIC |
| | NVNHKPSNTKVDKRVEPKSCDKTHTCPPCPAPELVGGPSVFLLPPKPKDTL |
| | MISRTPEVTCVVVDVSHEDPEVKFNWYVDGVEVHNAKTKPPEEQYNSTL |
| | RVVSVLTVLHQDWLNGKEYKCKVSNKALPAPIEKTISKAKGQPREPQVYT |
| | LPPSRDELTKNQVSLTCLVKGFYPSDIAVEWESNGQPENNYKTTPLVLDSD |
| | GSFFLYSKLTVDKSRWQQGNVFSCSVMHEALHNHYTQKSLSLSPGK (SEQ |
| | ID NO: 47) |
| Light Chain | DIVMTQSHKFMSTSVGDRVSITCKASQDVNTAVAWYQQKPGHSPKLLIYS |
| (LC) | ASFRYTGVPDRFTGSRSGTDFTFTISSVQAEDLAVYYCQQHYTTPPTFGGG |
| | TKVEIKRTVAAPSVFIFPPSDEQLKSGTASVVCLLNNFYPREAKVQWKVDN |
| | ALQSGNSQESVTEQDSKDSTYSLSSTLTLSKADYEKHKVYACEVTHQGLS |
| | SPVTKSFNRGEC (SEQ ID NO: 48) |

Avelumab and its methods of preparation can be found in U.S. Pat. No. 11,274,154, which is hereby incorporated by reference in its entirety.

Heavy Chain CDRs-Avelumab

| HCDR1 | HCDR2 | HCDR3 |
|---|---|---|
| SYIMM | SIYPSGGITFY | IKLGTVTTVDY |
| (SEQ ID NO: 49) | (SEQ ID NO: 50) | (SEQ ID NO: 51) |

Light Chain CDRs-Avelumab

| LCDR1 | LCDR2 | LCDR3 |
|---|---|---|
| TGTSSDVGGYNYVS | DVSNRPS | SSYTSSSTRV |
| (SEQ ID NO: 52) | (SEQ ID NO: 53) | (SEQ ID NO: 54) |

| | |
|---|---|
| Heavy Chain | EVQLLESGGGLVQPGGSLRLSCAASGFTFSSYIMMWVRQAPGKGLEWVS |
| (HC) | SIYPSGGITFYADTVKGRFTISRDNSKNTLYLQMNSLRAEDTAVYYCARIK |
| | LGTVTTVDYWGQGTLVTVSSASTKGPSVFPLAPSSKSTSGGTAALGCLVK |
| | DYFPEPVTVSWNSGALTSGVHTFPAVLQSSGLYSLSSVVTVPSSSLGTQTY |
| | ICNVNHKPSNTKVDKKVEPKSCDKTHTCPPCPAPELLGGPSVFLFPPKPKD |
| | TLMISRTPEVTCVVVDVSHEDPEVKFNWYVDGVEVHNAKTKPREEQYNS |
| | TYRVVSVLTVLHQDWLNGKEYKCKVSNKALPAPIEKTISKAKGQPREPQ |
| | VYTLPPSRDELTKNQVSLTCLVKGFYPSDIAVEWESNGQPENNYKTTPPV |
| | LDSDGSFFLYSKLTVDKSRWQQGNVFSCSVMHEALHNHYTQKSLSLSPG |
| | K (SEQ ID NO: 55) |
| Light Chain | QSALTQPASVSGSPGQSITISCTGTSSDVGGYNYVSWYQQHPGKAPKLMI |
| (LC) | YDVSNRPSGVSNRFSGSKSGNTASLTISGLQAEDEADYYCSSYTSSSTRVF |
| | GTGTKVTVLGQPKANPTVTLFPPSSEELQANKATLVCLISDFYPGAVTVA |
| | WKADGSPVKAGVETTKPSKQSNNKYAASSYLSLTPEQWKSHRSYSCQVT |
| | HEGSTVEKTVAPTECS (SEQ ID NO: 56) |

### Necitumumab

Necitumumab and its methods of preparation can be found in the art.

| | |
|---|---|
| Heavy Chain | QVQLQESGPGLVKPSQTLSLTCTVSGGSISSGDYYWSWIRQPPGKGLEWI |
| (HC) | GYIYYSGSTDYNPSLKSRVTMSVDTSKNQFSLKVNSVTAADTAVYYCAR |
| | VSIFGVGTFDYWGQGTLVTVSSASTKGPSVLPLAPSSKSTSGGTAALGCL |
| | VKDYFPEPVTVSWNSGALTSGVHTFPAVLQSSGLYSLSSVVTVPSSSLGT |
| | QTYICNVNHKPSNTKVDKRVEPKSCDKTHTCPPCPAPELLGGPSVFLFPPK |
| | PKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSL |
| | SPGK (SEQ ID NO: 57) |
| Light Chain | EIVMTQSPATLSLSPGERATLSCRASQSVSSYLAWYQQKPGQAPRLLIYD |
| (LC) | ASNRATGIPARFSGSGSGTDFTLTISSLEPEDFAVYYCHQYGSTPLTFGGGT |
| | KAEIKRTVAAPSVFIFPPSDEQLKSGTASVVCLLNNFYPREAKVQWKVDN |
| | ALQSGNSQESVTEQDSKDSTYSLSSTLTLSKADYEKHKVYACEVTHQGLS |
| | SPVTKSFNRGEC (SEQ ID NO: 58) |

### Trastuzumab

Trastuzumab and its methods of preparation can be found in the art.

| | |
|---|---|
| Heavy Chain | EVQLVESGGGLVQPGGSLRLSCAASGFNIKDTYIHWVRQAPGKGLEWVA |
| (HC) | RIYPTNGYTRYADSVKGRFTISADTSKNTAYLQMNSLRAEDTAVYYCSR |
| | WGGDGFYAMDYWGQGTLVTVSSASTKGPSVFPLAPSSKSTSGGTAALGC |
| | LVKDYFPEPVTVSWNSGALTSGVHTFPAVLQSSGLYSLSSVVTVPSSSLGT |
| | QTYICNVNHKPSNTKVDKKVEPKSCDKTHTCPPCPAPELLGGPSVFLFPPK |
| | PKDTLMISRTPEVTCVVVDVSHEDPEVKFNWYVDGVEVHNAKTKPREEQ |
| | YNSTYRVVSVLTVLHQDWLNGKEYKCKVSNKALPAPIEKTISKAKGQPR |
| | EPQVYTLPPSREEMTKNQVSLTCLVKGFYPSDIAVEWESNGQPENNYKTT |
| | PPVLDSDGSFFLYSKLTVDKSRWQQGNVFSCSVMHEALHNHYTQKSLSL |
| | SPG (SEQ ID NO: 59) |
| Light Chain | DIQMTQSPSSLSASVGDRVTITCRASQDVNTAVAWYQQKPGKAPKLLIYS |
| (LC) | ASFLYSGVPSRFSGSRSGTDFTLTISSLQPEDFATYYCQQHYTTPPTFGQGT |
| | KVEIKRTVAAPSVFIFPPSDEQLKSGTASVVCLLNNFYPREAKVQWKVDN |
| | ALQSGNSQESVTEQDSKDSTYSLSSTLTLSKADYEKHKVYACEVTHQGLS |
| | SPVTKSFNRGEC (SEQ ID NO: 60) |

Additional anti-EGFR antibodies, anti-HER-2 antibodies, anti-VEGFR2 antibodies, and anti-checkpoint inhibitor antibodies (for example anti-PD-L1 antibodies anti-PD-1 antibodies) are known in the art and can be obtained from commercial sources.

Strategies to Enhance NK Cell Response when Administered with Tumor-Targeting mAbs

A number of methods are known on the art to enhance NK cell response when administered in combination with mAbs targeting cancer antigens. These include genetic manipulation or glycoengineering of an antibody Fc region to modulate their interaction with activating and inhibitory members of the FcγR family. Examples include the A297N mutation used in atezolizumab, insertion of short sequences such as (GGGS) in the hinge region of the IgG1 heavy chain (e.g., between G237 and G238), and other changes, or the removal of fucose residues, increase IgG affinity for CD16 and result in enhanced NK cell-mediated ADCC and/or serial killing of multiple mAb-coated target cells. Another approach involves employing a multispecific antibody that targets two or more different epitopes of the same antigen.

NK Cell Engagers and Tri-specific Killer Cell Engagers (TriKEs). NK Cell engagers (NKCEs) are bispecific polypeptides that bind an NK cell surface antigen, e.g., CD16, and a cancer cell antigen, including without limitation, HER2, EGFR, or PD-L1, and induce NK cell cytotoxicity. In some embodiments, an NK Cell Engager is a bispecific monoclonal antibody. TriKEs are tri-specific polypeptides that bind an NK cell surface antigen, e.g., CD16, and two different cancer cell antigens, including without limitation, HER2, EGFR, CD33, or PD-L1, and induce NK cell cytotoxicity.

### Pharmaceutical Compositions and Treatment Methods

Also disclosed is a pharmaceutical, or immunotherapeutic, composition comprising a disclosed composition of matter in a pharmaceutically acceptable carrier. Pharmaceutical carriers are known to those skilled in the art. These most typically would be standard carriers for administration of drugs to humans, including solutions such as sterile water, saline, and buffered solutions at physiological pH. Typically, an appropriate amount of a pharmaceutically-acceptable salt is used in the formulation to render the formulation isotonic. Examples of the pharmaceutically-acceptable carrier include, but are not limited to, saline, Ringer's solution and dextrose solution. The pH of the solution is preferably from about 5 to about 8, and more preferably from about 7 to about 7.5. The solution should be RNAse free. Further carriers include sustained release preparations such as semipermeable matrices of solid hydrophobic polymers containing the antibody, which matrices are in the form of shaped articles, e.g., films, liposomes or microparticles. It will be apparent to those persons skilled in the art that certain carriers may be more preferable depending upon, for instance, the route of administration and concentration of composition being administered.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Compositions may be administered by, e.g., intravenous infusion or any other method appropriate for the delivery of living cells and antibody therapeutics (either together or separately).

### Treatment Applications

NK Cells disclosed herein can be used in the treatment or prevention of progression of proliferative diseases such as cancers and myelodysplastic syndromes. The cancer may be a hematologic malignancy or solid tumor.

Hematologic malignancies include leukemias, lymphomas, multiple myeloma, and subtypes thereof. Lymphomas can be classified various ways, often based on the underlying type of malignant cell, including Hodgkin's lymphoma (often cancers of Reed-Sternberg cells, but also sometimes originating in B cells; all other lymphomas are non-Hodgkin's lymphomas), non-Hodgkin's lymphomas, B-cell lymphomas, T-cell lymphomas, mantle cell lymphomas, Burkitt's lymphoma, follicular lymphoma, and others as defined herein and known in the art. Myelodysplastic syndromes comprise a group of diseases affecting immature leukocytes and/or hematopoietic stem cells (HSCs); MDS may progress to AML.

B-cell lymphomas include, but are not limited to, diffuse large B-cell lymphoma (DLBCL), chronic lymphocytic leukemia (CLL)/small lymphocytic lymphoma (SLL), and others as defined herein and known in the art.

T-cell lymphomas include T-cell acute lymphoblastic leukemia/lymphoma (T-ALL), peripheral T-cell lymphoma (PTCL), T-cell chronic lymphocytic leukemia (T-CLL), Sezary syndrome, and others as defined herein and known in the art.

Leukemias include acute myeloid (or myelogenous) leukemia (AML), chronic myeloid (or myelogenous) leukemia (CML), acute lymphocytic (or lymphoblastic) leukemia (ALL), chronic lymphocytic leukemia (CLL) hairy cell leukemia (sometimes classified as a lymphoma), and others as defined herein and known in the art.

Plasma cell malignancies include lymphoplasmacytic lymphoma, plasmacytoma, and multiple myeloma.

Solid tumors include melanomas, neuroblastomas, gliomas or carcinomas such as tumors of the brain, head and neck, breast, lung (e.g., non-small cell lung cancer, NSCLC), reproductive tract (e.g., ovary), upper digestive tract, gastroesophageal adenocarcinoma (GEA, also known as bowel cancer, colon cancer, or rectal cancer), pancreas, liver, renal system (e.g., kidneys), bladder, prostate and colorectum.

Head and neck cancers include squamous cell carcinoma of head and neck (SCCHN, a heterogeneous group of epithelial neoplasms that arise from upper aerodigestive tract), soft tissue sarcomas, nasopharyngeal cancer, laryngeal cancer, paransal sinus cancer, salivary gland cancer, and nasal cavity cancer.

Treatments and therapeutic (including immunotherapeutic) compositions and kits comprising memory NK cells and mAbs disclosed herein may be administered in any order or contemporaneously.

Methods described herein are generally performed on a subject in need thereof. A subject in need of the therapeutic methods described herein can be a subject having, diagnosed with, suspected of having, or at risk for developing, or at rick of progressing to a later stage of, cancer. A determination of the need for treatment will typically be assessed by a history, physical exam, or diagnostic tests consistent with the disease or condition at issue. Diagnosis of the various conditions treatable by the methods described herein is within the skill of the art. The subject can be an animal subject, including a mammal, such as horses, cows, dogs, cats, sheep, pigs, mice, rats, monkeys, hamsters, guinea pigs, and humans, or other animals such as chickens. For example, the subject can be a human subject. Generally, a safe and effective amount of a therapy, e.g. a memory NK cell in combination with an anti-cancer monoclonal antibody is, for example, an amount that would cause the desired therapeutic effect in a subject while minimizing undesired side effects.

According to the methods described herein, administration can be parenteral, pulmonary, oral, topical, intradermal, intramuscular, intraperitoneal, intravenous, intratumoral, intrathecal, intracranial, intracerebroventricular, subcutaneous, intranasal, epidural, ophthalmic, buccal, or rectal administration. Where the product is, for example, a biologic or cell therapy, the mode of administration will likely be via intravenous injection or infusion.

### Definitions

Unless otherwise defined, scientific and technical terms used in connection with the present disclosure shall have the meanings that are commonly understood by those of ordinary skill in the art. Further, unless otherwise required by context, singular terms shall include pluralities and plural terms shall include the singular. Generally, nomenclatures utilized in connection with, and techniques of, cell and tissue culture, molecular biology, and protein and oligo or polynucleotide chemistry and hybridization described herein are those well-known and commonly used in the art.

As used herein, the term “antibody” refers to a polypeptide that includes canonical immunoglobulin sequence elements sufficient to confer specific binding, or e.g. immune-reacts and/or is directed to a particular target antigen. As is known in the art, intact antibodies as produced in nature are approximately 150 kD tetrameric agents comprised of two identical heavy chain polypeptides (about 50 kD each) and two identical light chain polypeptides (about 25 kD each) that associate with each other into what is commonly referred to as a “Y-shaped” structure. Each heavy chain is comprised of at least four domains (each about 110 amino acids long)—an amino-terminal variable (V_{H}) domain, followed by three constant domains: C_{H}1, C_{H}2, and the carboxy-terminal C_{H}3. A short region, known as the “switch”, connects the heavy chain variable and constant regions. The “hinge” connects C_{H}2 and C_{H}3 domains to the rest of the antibody. Two disulfide bonds in this hinge region connect the two heavy chain polypeptides to one another in an intact antibody. Each light chain is comprised of two domains—an amino-terminal variable (V_{L}) domain, followed by a carboxy-terminal constant (C_{L}) domain, separated from one another by another “switch”. Intact antibody tetramers are comprised of two heavy chain-light chain dimers in which the heavy and light chains are linked to one another by a single disulfide bond; two other disulfide bonds connect the heavy chain hinge regions to one another, so that the dimers are connected to one another and the tetramer is formed. Naturally-produced antibodies are also glycosylated, typically on the C_{H}2 domain. Each domain in a natural antibody has a structure characterized by an “immunoglobulin fold” formed from two beta sheets (e.g., 3-, 4-, or 5-stranded sheets) packed against each other in a compressed antiparallel beta barrel. Each variable domain contains three hypervariable loops known as “Complementarity-Determining Regions” (CDR1, CDR2, and CDR3) and four somewhat invariant “framework” regions (FR1, FR2, FR3, and FR4). When natural antibodies fold, the FR regions form the beta sheets that provide the structural framework for the domains, and the CDR loop regions from both the heavy and light chains are brought together in three-dimensional space so that they create a single hypervariable antigen binding site located at the tip of the Y structure. The Fc region of naturally-occurring antibodies binds to elements of the complement system, and also to receptors on effector cells, including for example effector cells that mediate cytotoxicity.

An “antibody fragment” refers to a molecule other than an intact antibody that comprises a portion of an intact antibody that binds the antigen to which the intact antibody binds. Several examples of antibody fragments include but are not limited to Fv, Fab, Fab′, Fab′-SH, F(ab′)2, diabodies, linear antibodies, single chain variable fragments (scFvs), and multi-specific antibodies formed from antibody fragments. In some embodiments, the antibody fragment is an antigen-binding fragment.

Reviews of current methods for antibody engineering and improvement can be found in R. Kontermann and S. Dubel, (2010) Antibody Engineering Vols. 1 and 2, Springer Protocols, 2nd Edition and W. Strohl and L. Strohl (2012) Therapeutic antibody engineering: Current and future advances driving the strongest growth area in the pharmaceutical industry, Woodhead Publishing. Methods for producing and purifying antibodies and antigen-binding fragments are well known in the art and can be found, in Harlow and Lane (1988) Antibodies: A Laboratory Manual, Cold Spring Harbor Laboratory Press, Cold Spring Harbor, NY, chapters 5-8 and 15.

The term “antigen” refers to a molecular entity that may be soluble or cell membrane bound in particular but not restricted to molecular entities that can be recognized by means of the adaptive immune system including but not restricted to antibodies or TCRs, or engineered molecules including but not restricted to transgenic TCRs, chimeric antigen receptors (CARs), scFvs or multimers thereof, Fab-fragments or multimers thereof, antibodies or multimers thereof, single chain antibodies or multimers thereof, or any other molecule that can execute binding to a structure with high affinity.

As used herein, the term “binding affinity” refers to the strength of binding of one molecule to another at a site on the molecule. If a particular molecule will bind to or specifically associate with another particular molecule, these two molecules are said to exhibit binding affinity for each other. Binding affinity is related to the association constant and dissociation constant for a pair of molecules, but it is not critical to the methods herein that these constants be measured or determined. Rather, affinities as used herein to describe interactions between molecules of the described methods are generally apparent affinities (unless otherwise specified) observed in empirical studies, which can be used to compare the relative strength with which one molecule (e.g., an antibody or other specific binding partner) will bind two other molecules (e.g., two versions or variants of a peptide). The concepts of binding affinity, association constant, and dissociation constant are well known.

The term “cancer” is known medically as a malignant neoplasm. Cancer is a broad group of diseases involving upregulated cell growth. In cancer, cells (cancerous cells) divide and grow uncontrollably, forming malignant tumors, and invading nearby parts of the body. The cancer may also spread to more distant parts of the body through the lymphatic system or bloodstream. There are over 200 different known cancers that affect humans.

The term “chemotherapy” refers to the treatment of cancer (cancerous cells) with one or more cytotoxic anti-neoplastic drugs (“chemotherapeutic agents” or “chemotherapeutic drugs”) as part of a standardized regimen. Chemotherapy may be given with a curative intent or it may aim to prolong life or to palliate symptoms. It is often used in conjunction with other cancer treatments, such as radiation therapy, surgery, and/or hyperthermia therapy. Traditional chemotherapeutic agents act by killing cells that divide rapidly, one of the main properties of most cancer cells. This means that chemotherapy also harms cells that divide rapidly under normal circumstances, such as cells in the bone marrow, digestive tract, and hair follicles. This results in the most common side-effects of chemotherapy, such as myelosuppression (decreased production of blood cells, hence also immunosuppression), mucositis (inflammation of the lining of the digestive tract), and alopecia (hair loss).

The term “combination immunotherapy” refers to the concerted application of two therapy approaches e.g. therapy approaches known in the art for the treatment of disease such as cancer. The term “combination immunotherapy” may also refer to the concerted application of an immunotherapy such as the treatment with an antigen recognizing receptor and another therapy such as the transplantation of NK cells e.g. memory NK cells. Expression of an antigen on a cell means that the antigen is sufficient present on the cell surface of the cell, so that it can be detected, bound and/or recognized by an antigen-recognizing receptor.

The term “cytokine-induced memory-like,” or, equivalently, “CIML,” in reference to NK cells, means having a “memory” or “memory-like” phenotype and produced using a priming agent.

The term “cytotoxicity,” as used herein in reference to memory NK cells, refers to the ability of cells to target and kill diseased cells.

A “diseased cell” refers to the state of a cell, tissue or organism that diverges from the normal or healthy state and may result from the influence of a pathogen, a toxic substance, irradiation, or cell internal deregulation. A “diseased cell” may also refer to a cell that has been infected with a pathogenic virus. Further the term “diseased cell” may refer to a malignant cell or neoplastic cell that may constitute or give rise to cancer in an individual.

The terms “engineered cell” and “genetically modified cell” as used herein can be used interchangeably. The terms mean containing and/or expressing a foreign gene or nucleic acid sequence, or containing a gene which has been genetically modified to deviate from its natural form or function (for example a deleted or knocked-out gene) which in turn modifies the genotype or phenotype of the cell or its progeny. Cells can be modified by recombinant methods well known in the art to express stably or transiently peptides or proteins, which are not expressed in these cells in the natural state. Methods of genetic modification of cells may include but is not restricted to transfection, electroporation, nucleofection, transduction using retroviral vectors, lentiviral vectors, non-integrating retro-or lentiviral vectors, transposons, designer nucleases including zinc finger nucleases, TALENs or CRISPR/Cas nucleases.

The term “enrich” as used herein in relation to NK cells means to concentrate, purify, or isolate for further analysis or use. Enriched and purified cell populations comprise a majority of the desired cell, and a negligible fraction of other cells.

The term “fold selective,” as used herein, means having an affinity for one target that is at least x-fold greater than its affinity for another target, wherein x is at least 2, and may be higher, e.g., 10, 20, 50, 100, or 1000. In preferred embodiments, the fold selectivity is therapeutically meaningful, i.e., sufficient to permit cells expressing one target to be killed and cells bearing the other target to be spared.

The term “genetic modification” or genetically modified” refers to the alteration of the nucleic acid content including but not restricted to the genomic DNA of a cell. This includes but is not restricted to the alteration of a cells genomic DNA sequence by introduction exchange or deletion of single nucleotides or fragments of nucleic acid sequence. The term also refers to any introduction of nucleic acid into a cell independent of whether that leads to a direct or indirect alteration of the cells genomic DNA sequence or not.

The term “immune cell” or “immune effector cell” refers to a cell that may be part of the immune system and executes a particular effector function such as alpha-beta T cells, NK cells (including memory NKs, ML-NKs, and CIML-NKs), NKT cells (including iNKT cells), B cells, innate lymphoid cells (ILC), cytokine induced killer (CIK) cells, lymphokine activated killer (LAK) cells, gamma-delta T cells, mesenchymal stem cells or mesenchymal stromal cells (MSC), monocytes and macrophages. Preferred immune cells are cells with cytotoxic effector function such as alpha-beta T cells, NK cells (including memory NKs, ML-NKs, and CIML-NKs), NKT cells (including iNKT cells), ILC, CIK cells, LAK cells or gamma-delta T cells. “Effector function” means a specialized function of a cell, e.g. in an NK cell an effector function may be cytolytic activity or helper activity including the secretion of cytokines.

The term “immunotherapy” is a medical term defined as the “treatment of disease by inducing, enhancing, or suppressing an immune response” Immunotherapies designed to elicit or amplify an immune response are classified as activation immunotherapies, while immunotherapies that reduce or suppress are classified as suppression immunotherapies. Cancer immunotherapy as an activating immunotherapy attempts to stimulate the immune system to reject and destroy tumors. Adoptive cell transfer uses cell-based cytotoxic responses to attack cancer cells Immune cells such as T cells that have a natural or genetically engineered reactivity to a patient's cancer are generated in vitro and then transferred back into the cancer patient.

As used herein, the term “individual” refers to an animal. Preferentially, the individual is a mammal such as mouse, rat, cow, pig, goat, chicken dog, monkey or human. More preferentially, the individual is a human. The individual may be an individual suffering from a disease such as cancer (a patient), but the subject may be also a healthy subject. The term “malignant” or “malignancy” describes cells, groups of cells or tissues that constitute a neoplasm, are derived from a neoplasm or can be the origin of new neoplastic cells. The term is used to describe neoplastic cells in contrast to normal or healthy cells of a tissue. A malignant tumor contrasts with a non-cancerous benign tumor in that a malignancy is not self-limited in its growth, is capable of invading into adjacent tissues, and may be capable of spreading to distant tissues. A benign tumor has none of those properties. Malignancy is characterized by anaplasia, invasiveness, and metastasis as well as genome instability. The term “premalignant cells” refer to cells or tissue that is not yet malignant but is poised to become malignant.

The term “memory” or “memory-like,” in reference to NK cells, means having an activated phenotype with improved cytotoxicity and longevity/persistence compared to a general population of NK cells, and typically exhibits increased cell-surface expression of CD69, CD25, and NKG2A, and maintained expression of CD16, compared to a general population of NK cells.

The term “monoclonal antibody” (mAb), as applied to the antibodies described in the present disclosure, are compounds derived from a single copy or a clone from any eukaryotic, prokaryotic, or phage clone, and not the method by which it is produced. mAbs of the present disclosure may exist in a homogeneous or substantially homogeneous population.

The term “persistence” as sued herein refers to the ability of cells, especially adoptively transferred into a subject, to continue to live.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

The term “prime,” in reference to NK cells, means to stimulate or activate into a memory/memory-like phenotype using a priming agent. A “priming agent” comprises a combination of stimulatory cytokines, for example,
one or more of IL-12, IL-23, IL-27, and IL-35;
one or more of IL-2, IL-4, IL-7, IL-9, IL-15, and IL-21; and
one or more of IL-18, IL-1a, IL-1b, IL-36a, IL-36b, and IL-36g,
   or one or more “priming fusion proteins” comprising functional fragments of such cytokines, or one or more multichain complexes thereof. Examples of such proteins are disclosed herein.

In general, the term “receptor” refers to a biomolecule that may be soluble or attached to the cell surface membrane and specifically binds a defined structure that may be attached to a cell surface membrane or soluble. Receptors include but are not restricted to antibodies and antibody like structures, adhesion molecules, transgenic or naturally occurring TCRs or CARs. The term “antigen-recognizing receptor” or “antigen-binding receptor” as used herein may be a membrane bound or soluble receptor such as a natural TCR, a transgenic TCR, a CAR, a scFv or multimers thereof, a Fab-fragment or multimers thereof, an antibody or multimers thereof, a bi-specific T cell enhancer (BiTE), a diabody, or any other molecule that can execute specific binding with high affinity.

The terms “specifically binds” or “specific for” or “specifically recognize” with respect to an antigen-recognizing receptor refer to an antigen-binding domain of the antigen-recognizing receptor which recognizes and binds to a specific polymorphic variant of an antigen, but does not substantially recognize or bind other variants.

The term “side-effects” refers to any complication, unwanted or pathological outcome of an immunotherapy with an antigen recognizing receptor that occurs in addition to the desired treatment outcome. The term “side effect” preferentially refers to on-target off-tumor toxicity, that might occur during immunotherapy in case of presence of the target antigen on a cell that is an antigen-expressing non-target cell but not a diseased cell as described herein. A side-effect of an immunotherapy may be the developing of graft versus host disease.

The term “target” or “target antigen” refers to any cell surface protein, glycoprotein, glycolipid or any other structure present on the surface of the target cell. The term also refers to any other structure present on target cells in particular but not restricted to structures that can be recognized by means of the adaptive immune system including but not restricted to antibodies or TCRs, or engineered molecules including but not restricted to transgenic TCRs, CARs, scFvs or multimers thereof, Fab-fragments or multimers thereof, antibodies or multimers thereof, single chain antibodies or multimers thereof, or any other molecule that can execute binding to a structure with high affinity.

The term “target cells” as used herein refers to cells which are recognized by the antigen-recognizing receptor which is or will be applied to the individual.

The term “therapeutically effective amount” means an amount which provides a therapeutic benefit.

As used herein, the term “transplant” means administering to a subject a population of donor cells, e.g. NK cells.

The term “treatment” as used herein means to reduce the frequency or severity of at least one sign or symptom of a disease.

### EXAMPLES

The invention is further illustrated by the following examples.

### Example 1. In Vitro Culturing of Memory NK Cells

Materials and Methods: Memory NK cells may be produced as described in disclosed in WO2020047299, WO202047473, or WO 2020257639, or by methods known on the art.

Alternatively, Memory NK cells may be produced as follows. NK cells may be isolated from whole blood using CD3 depletion and CD56 positive selection. NK cells selected are then cultured in 96-well plates in NK MACS media+supplements+10% HI-HAB, and primed/expanded in the following conditions, where 1× 7t15-21s and ATF1 is 200 nM and 100 nM respectively, and 1× 18t15-12s is 250 nM; all dilutions are calculated from these values as indicated.
a) Expand Only: +7t15-21s and ATF1 for either 2, 6, or 10 days in 37deg, 5% CO2 at the indicated concentrations. Every 2 days following, 7t15-21s and ATF1 are replenished to the indicated concentrations with fresh media.
b) Prime and Expand: +18t15-12s for either 2, 6, or 10 days in 37deg, 5% CO2 at the indicated concentrations. Every 2 days following, 7t15-21s and ATF1 are replenished to the indicated concentrations with fresh media.

### Example 2. Phenotyping of Memory NK Cells

To assess the phenotype of the NK cells generated by the above processes, at the appropriate timepoint, NK cells are harvested, washed, and assessed for receptor expression by staining with a flow panel comprising purity and/or activation markers, for example, anti-CD56, anti-CD3, Live/Dead Yellow, anti-NKG2A, anti-CD69, anti-CD25, and anti-CD16. The following clones are used:
Anti-CD45 (HI30 clone)
Anti-CD56 (CMSSB clone)
Anti-CD3 (SK7 clone)
Live/Dead Yellow (Thermo Fisher)
anti-NKG2A (REA110 clone)
anti-CD69 (FN50 clone)
anti-CD25 (CD25-4E3 clone)
anti-CD16 (eBioCD16 clone)

An Attune NXt flow cytometer is used. Data are then analyzed in Flowjo v10.7, gating on Live CD56+CD3− cells and assessing the median fluorescence intensity of each of the above described markers. Increases in CD69, CD25, and NKG2A expression, and maintenance of CD16 expression, indicates a memory-NK cell phenotype.

### Example 3. ADCC of Memory NK Cells

To assess killing activity of the NK cells generated by the above processes, at the appropriate timepoint, Cultured NK cells are harvested and washed, then resuspended in NK MACS Media with 10% human AB serum (Gibco)) and may be added to a 96-well plate with 10,000 Luciferase expressing human tumor cells (ATCC) at the indicated effector to target (E:T) ratios 24-48 hours, with or without IL-2 (Miltenyi), after which luciferase activity (live tumor cells) is assessed by luciferase readout (Promega). Data not shown.

Result: This example may be used to demonstrate the in vitro activity and flow cytometry phenotype of NK cells generated by the above processes. Cumulative fold change in surface protein expression, cell size, and median fluorescence intensities for individual genes may be tracked. Memory NK cells exhibit increased ADCC compared to normal NK cells.

### Example 4. In Vitro ADCC Cytotoxicity Assay Using IncuCyte

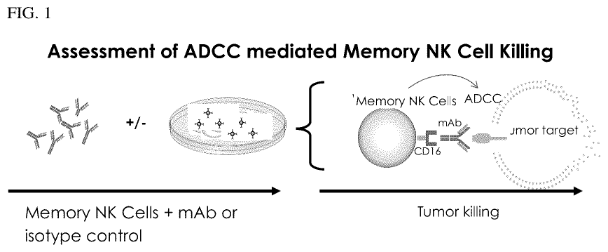

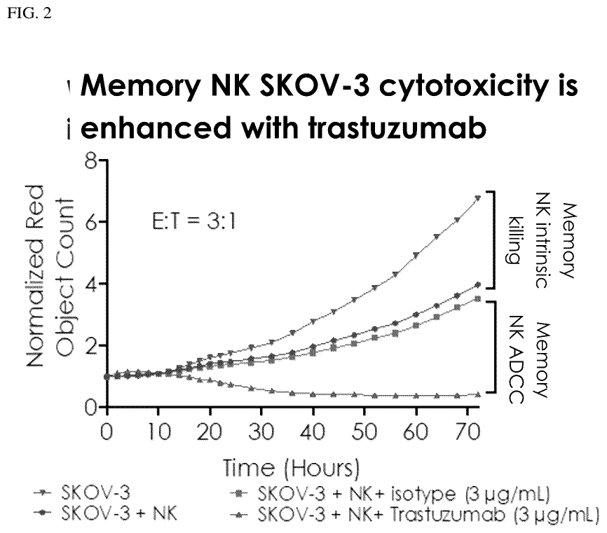

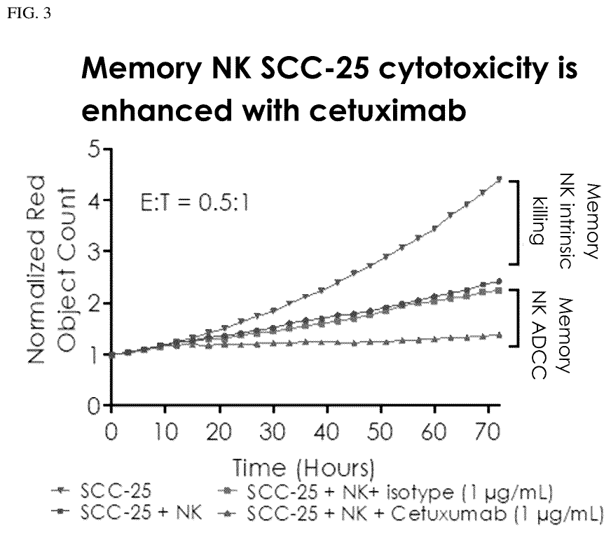

Head and neck cancer cell line (SCC-25) or ovarian cell line (SKOV-3) were transduced with red fluorescent protein using the NucLight Red lentiviral reagent (NLR; Essen BioScience). For kinetic analysis of tumor cell killing, each NLR-transduced cell line was individually plated in 96-well flat-bottom plates at a concentration of 3×10³ or 4×10³ cells per well, respectively, and incubated in humidified incubator overnight. The following day, NK cells were added at Effector:Target (E:T) ratios ranging from 0.5:1 to 10:1. Cetuximab and Trastuzumab were also added at the concentration of 1 and 3 ug/ml, respectively. All cocultures were performed in respective target cell medium and 100U/ml human IL-2. Live-cell numbers were monitored by frequent fluorescence imaging (every 3 hours) for 72 hours, quantified by IncuCyte S3 software (Essen BioScience) and normalized to the number of live cells remaining in the target cell-only control group. Results are shown in FIGS. 1, 2, and 3.

### Example 5. Memory NK Cell Persistence and Surface Expression of CD16

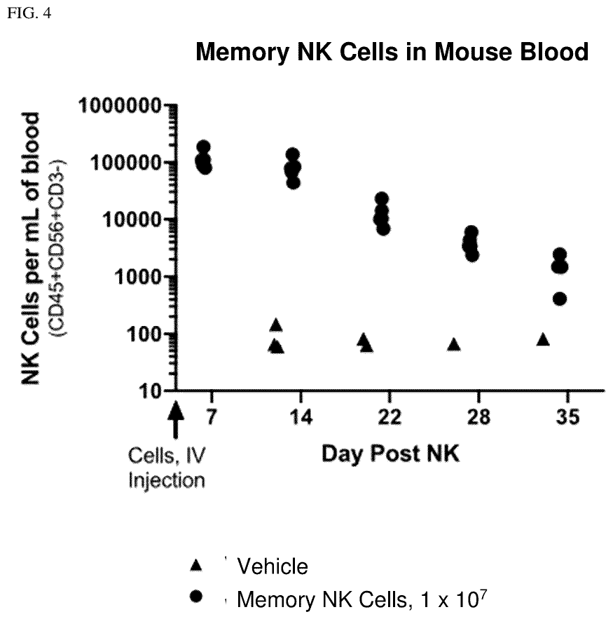

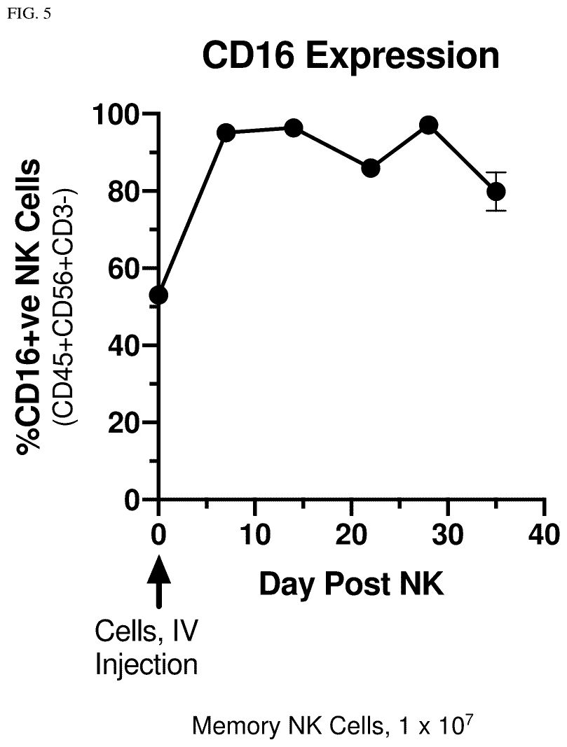

The anti-tumor activity and persistence of memory NK cells were assessed in a model of acute myeloid leukemia (AML) using THP-1 cells model. THP-1 cells (ATCC TIB-202) were engineered to express green fluorescent protein (GFP) and click-beetle red luciferase (CBR) to allow monitoring of tumor burden by measuring whole body bioluminescence (BLI) using an IVIS imager. THP-1-CBR-GFP cells (2×10′ cells/mouse) were inoculated in female NSG mice (Jackson Labs; N=5/group) via i.v. injection into the tail vein. Tumors were allowed to engraft for 3 days prior to treatment with vehicle or memory NK Cells (1×10⁷ cells/mouse) via tail vein injection. Tumor progression was followed by whole body bioluminescence (BLI) measurements under isoflurane anesthesia. Blood samples, obtained by submandibular vein bleeds using EDTA anticoagulant, were incubated with the appropriate antibody fluorophore conjugates [CD16 PerCP-eF710 (ThermoFisher 46-0168-42), CD45 APC-eF780 (ThermoFisher 47-0459-42), mCD45 BV421 (BD Biosciences 563890), Viability Live/Dead Yellow (ThermoFisher L34968), CD3 SB780 (ThermoFisher 78-0036-42), CD56 PE-Cy7 (ThermoFisher 25-0567-42), mouse CD16/32 Fc Block (BioLegend 156604)] for 30 minutes on ice in staining buffer (DPBS+2% HI-FBS+2 mM EDTA). Stained blood samples were washed with staining buffer and then incubated with fixation/permeabilization buffer (ThermoFisher 00-5123-43) to lyse red blood cells and fix samples. After lysis, samples were washed with staining buffer and data was acquired on an Attune NxT Flow Cytometer (ThermoFisher). Analyses were performed on FCS Express 7 software. Population densities were quantified to cells/mL of blood using in sample CountBright Absolute Counting Beads (ThermoFisher C36950). Results are shown in FIGS. 4 and 5.

CD16 (FcγRIII) is the Fc receptor that binds the Fc domain of monoclonal antibodies to immune cells to enable cell-associated antibody-dependent cellular cytotoxicity (ADCC) activity, leading to enhanced tumor killing. CD16 expression on the surface of memory NK cells increases from the time of injection into mice and persists throughout the course if of the study. This enables the binding of the Fc domain of monoclonal antibodies to memory NK cells and enhances the ADCC activity of memory NK cell therapy.

### Example 6. IFNg Induces PD-L1 Expression on SCC-25 Head & Neck Cancer Cells

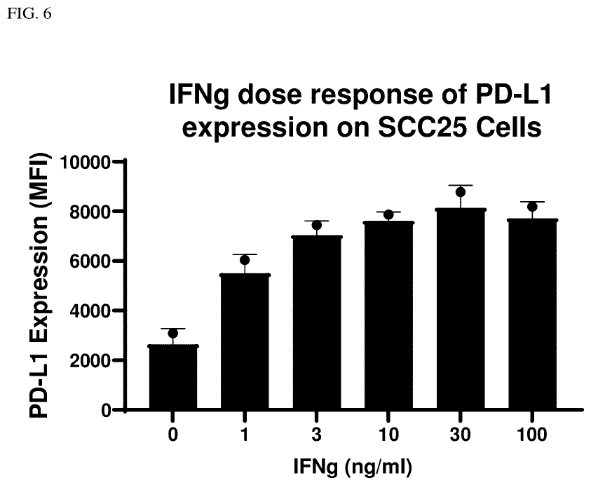

SCC-25 cells were cultured in DMEM/F12 media containing 10% FBS, 1% PenStrep, 1% sodium bicarbonate, 1% HEPES, 1% glutamax, 1% sodium pyruvate. Cells were incubated with increasing concentrations of recombinant human IFNg (R&D Systems; Cat #285-IF-100), and incubated for 24 hours at 37° C. The cells were then harvested, washed and PD-L1 expression was determined by FACS analysis using PE anti-human CD274 (B7-H1, PD-L1) Antibody (BioLegend Cat #393608) and Attune NxT Flow Cytometer, Life Technologies. PD-L1 expression on SCC-25 cells is expressed as a mean fluorescent intensity (MFI). Results are shown in FIG. 6.

### Example 7. IFNg Induces PD-L1 Expression on LoVo Colon Cancer Cells

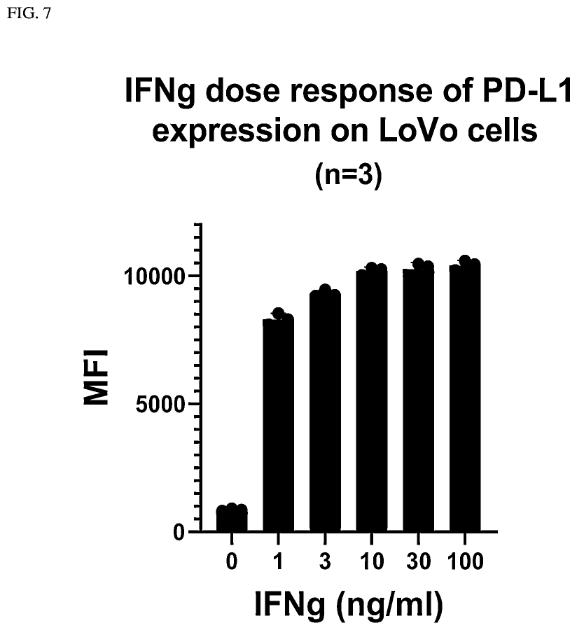

LoVo cells were cultured in F12K media containing 10% FBS, 1% PenStrep. Cells were treated with increasing concentrations of recombinant human IFNg (R&D Systems; Cat #285-IF-100) and incubated for 24 hours at 37° C. The cells were harvested, washed and PD-L1 expression was determined by FACS analysis using PE anti-human CD274 (B7-H1, PD-L1) Antibody (BioLegend Cat. 393608) and Attune NxT Flow Cytometer, Life Technologies. A fluorophore- and isotype-matched mouse antibody (BioLegend Cat. 400113) was used as a negative control for FACS analysis. Results are shown in FIG. 7.

### Example 8. Memory NK Cells Killing of Tumor Cells Increases Surface PD-L1 Expression, Opening the Opportunity for Checkpoint Inhibitor (CPI) Combination

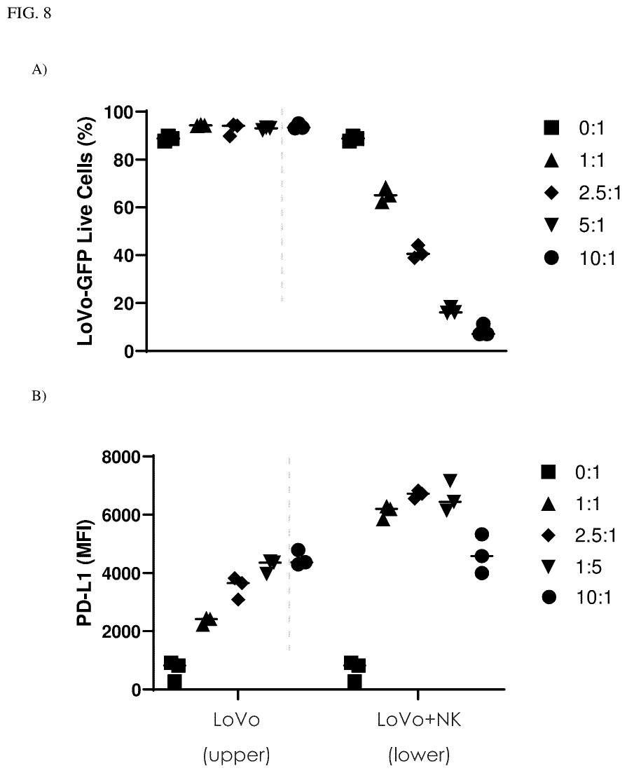

Using a transwell assay (Corning® HTS Transwell® 24 well-plate Cat #3422, 8 um pore size), we have tested the killing of solid tumor cells, such as colorectal cancer cell line (LoVo), and in the same experiment we measure the expression of PD-L1 on LoVo cells. First, we co-cultured LoVo-GFP with memory NK cells in the lower chamber in different ratios (Effector:Target (E:T) 0:1, 1:1, 2.5:1, 5:1, 10:1 with a target cell number fixed at 25×10³ in 0.6 mL culture media); then the upper chamber containing 25×10³ LoVo cells in 100 uL was placed inside that well. After 24 hours, the cells from the upper chamber and lower chamber were harvested, washed, and stained with anti-human PD-L1 antibody (PE, Cat #393608, clone MIH2) and the Live-Dead Fixable Far-Red dye (Cat #L34974) for 30 minutes on ice. The cells were washed and resuspended in 200 uL FACS buffer and analyzed by flow cytometer (Attune NxT Flow Cytometer, Life Technologies). The tumor cell viability is expressed as the % of Live LoVo-GFP+ cells. The PD-L1 expression on LoVo cells is expressed as a mean fluorescent intensity (MFI). Results are shown in FIG. 8.

These findings suggest that the increasing ratio of memory cells enhances killing of LoVo cells in the lower chamber and secretes higher levels of cytokines such as interferon gamma (IFN-γ), which in turn stimulates the surface expression of PD-L1 in LoVo cells in the upper chamber. Memory NK-mediated PD-L1 expression on cancer cells opens up the opportunity for combination treatment with checkpoint inhibitors (CPI) such as anti-PD1 and anti-PDL1 antibodies.

### Example 9. Combination of Memory NK Cells with ADCC-Capable mAbs Further Enhances PD-L1 Expression on Tumor Cells

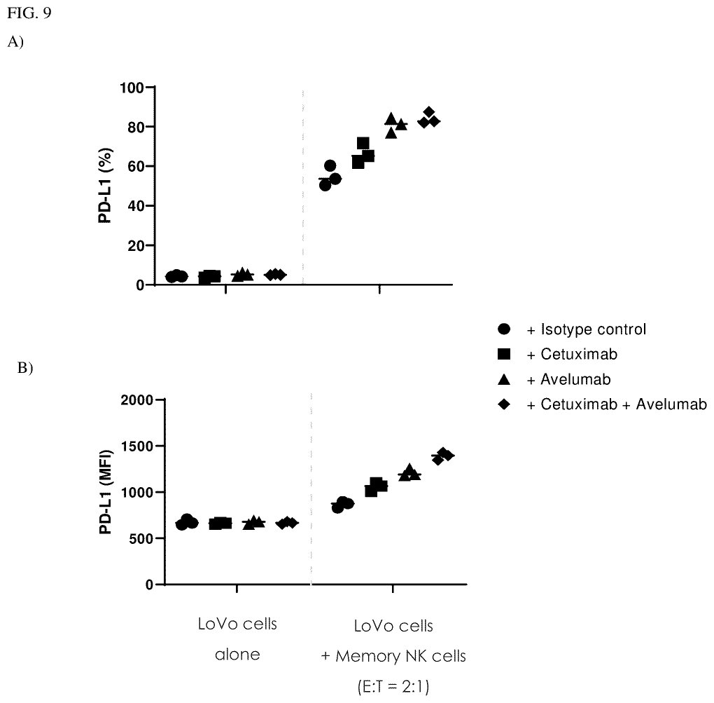

Using a transwell assay, we co-cultured LoVo-GFP with memory NK cells in the lower chamber in a ratio E:T 2:1 (50×10³ Memory NK cells: 25×10³ LoVo cells in 0.6 mL complete culture media); then we placed inside that well the upper chamber containing 25×10³ LoVo cells in 100 uL. To assess the impact of ADCC-capable mAbs on PD-L1 expression, we added 1 ug/ml of Cetuximab (an anti-EGFR antibody) or Avelumab (an anti-PD-L1 antibody), or a combination of Cetuximab and Avelumab to the culture media of the lower wells. After 48 hours, we harvested LoVo cells from the upper chamber, washed and stained with anti-human PD-L1 antibody (Brilliant Violet 711, Cat #329722, clone 29E.2A3) for 30 minutes on ice. Then, the cells were washed and resuspended in 200 uL FACS buffer, and the samples were analyzed by a flow cytometer (Attune NxT Flow Cytometer, Life Technologies). PD-L1 expression on LoVo cells in the upper chamber is demonstrated as a percentage (%) of PD-L1 positive cells and as a mean fluorescent intensity (MFI). Results are shown in FIG. 9.

These findings demonstrate that memory NK cells killing of LoVo cells in the lower chamber released soluble factors, most likely IFNg, into the culture media which increased the expression of PD-L1 on LoVo cells in the upper chamber, and that this was enhanced by the addition of Cetuximab, Avelumab, or the combination of both antibodies.

### Example 10. PD-L1 is Induced on SCC-25 Cells (Head and Neck Cancer) after Incubation with Memory NK Cells or Incubation with Conditioned Media

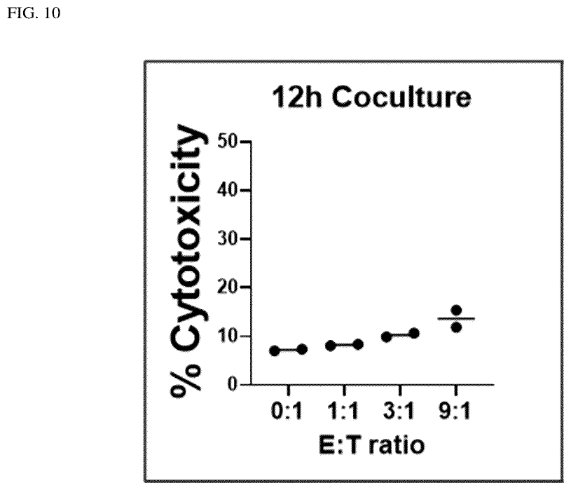

SCC-25 cells were co-cultured with memory NK cells for 12 h, 24 h, or 42 h at 37° C. and at E:T ratios of 0:1, 1:1, 3:1, or 9:1. At each time point, the supernatant was collected, spun at an elevated speed to eliminate any residual cells or cell debris, and transferred to wells containing fresh SCC-25 cells for the secondary conditioned cultures. The remaining cells of the primary cocultures were enzymatically harvested and used for flow cytometry analysis to assess tumor cell death and surface PD-L1 expression by staining with Fixable Aqua Dead Cell Stain (Invitrogen Cat. L34957A) and PE anti-human antibody (BioLegend Cat. 393608), respectively. The secondary conditioned cultures were allowed to grow for 24 hours, after which the cells were harvested, and analyzed in the same manner as the primary cocultures. Results are shown in FIG. 10.

From the primary cocultures, tumor cell death occurred in all the treatments in which memory NK cells were present, showing a positive relationship with the PD-L1 expression. Both cell death and PD-L1 expression were in a positive coordination with the coculturing duration and the ET ratio. However, in the conditioned cultures, where fresh SCC-25 cells were stimulated with soluble factors, including IFNg, present in the primary culture supernatant, only PD-L1 expression, but not cell death, showed a similar correlation as primary cocultures, indicating that IFNg does not cause tumor cell death. Taken together, these results indicate that memory NK cell-induced cell death induces PD-L1 expression on tumor cells, which supports the rationale of using anti-PDL1 antibodies such as avelumab for ADCC in combination with memory NKs and where Avelumab additionally disrupts the interaction of PD1 on T-cells and PDL-1 on tumor cells, enhancing T-cell-mediated immunotherapy.

### Example 11. Addition of Avelumab to NCI-N87 Cells (Gastric Cancer Cells)+Memory NK Cells Coculture Enhances the Killing of NCI-N87 Cells by an ADCC Mechanism

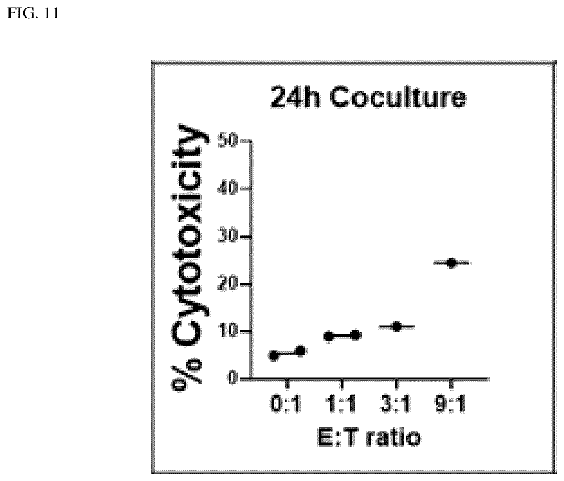

Fluorescently labeled gastric carcinoma cell line (NCI-N87), pretreated overnight with 30 ng/ml human recombinant IFNg to upregulate PD-L1, were either cultured alone or with memory NK cells (E:T=1:1) in the presence or absence of 1 ug/ml of Avelumab for 72 hours at 37° C. Tumor cell death, indicated as the disappearance of the live fluorescent tumor cells, was monitored via IncuCyte S3 Live-Cell Analysis Instrument. Results are shown in FIG. 11.

While Avelumab alone did not induce significant tumor cell death, addition of Avelumab to memory NK cells+NCI-N87 coculture cells significantly enhanced tumor cell death, indicating an ADCC-mediated tumor killing.

The detailed description set-forth above is provided to aid those skilled in the art in practicing the present invention. However, the invention described and claimed herein is not to be limited in scope by the specific embodiments herein disclosed because these embodiments are intended as illustration of several aspects of the invention. Any equivalent embodiments are intended to be within the scope of this invention. Indeed, various modifications of the invention in addition to those shown and described herein will become apparent to those skilled in the art from the foregoing description, which do not depart from the spirit or scope of the present inventive discovery. Such modifications are also intended to fall within the scope of the appended claims.

1.-106. (canceled)

107. A method of treating cancer in a subject in need thereof comprising administering memory natural killer (NK) cells and a monoclonal antibody.

108. The method of claim 107, wherein the monoclonal antibody is an Immunoglobulin G (IgG) antibody.

109. The method of claim 108, wherein the monoclonal antibody is chosen from an IgG1, IgG3, and IgG4 subtype.

110. The method of claim 109, wherein the Fc moiety of the monoclonal antibody is mutated to enhance CD16 binding affinity.

111. The method of claim 109, wherein the monoclonal antibody is chosen from an anti-HER2 antibody, an anti-PD-1 antibody, an anti-PDL-1 antibody, or an anti-EGFR antibody.

112. The method of claim 111, wherein the monoclonal antibody is an anti-EGFR monoclonal antibody.

113. The method of claim 112, wherein the cancer is head and neck cancer.

114. The method of claim 112, wherein the cancer is colorectal cancer.

115. The method of claim 112, wherein the anti-EGFR monoclonal antibody is chosen from cetuximab, panitumumab, and necitumumab.

116. The method of claim 115, wherein the anti-EGFR monoclonal antibody is cetuximab.

117. The method of claim 111, wherein the monoclonal antibody is an immune-checkpoint inhibitor.

118. The method of claim 117, wherein the immune-checkpoint inhibitor is chosen from an anti-PD-1 monoclonal antibody, an anti-PD-L1 monoclonal antibody, and an anti-CTLA-4 monoclonal antibody.

119. The method of claim 117, wherein the cancer is chosen from urothelial carcinoma, head and neck cancer, colorectal cancer, and gastric cancer.

120. The method of claim 118, wherein the antibody is an anti-PD-L1 monoclonal antibody.

121. The method of claim 120, wherein the anti-PD-L1 monoclonal antibody is chosen from atezolizumab, avelumab, coseibelimab, and durvalumab.

122. The method of claim 121, wherein the anti-PD-L1 monoclonal antibody is avelumab.

123. The method of claim 118, wherein the antibody is an anti-PD-1 monoclonal antibody.

124. The method of claim 123, wherein the anti-PD-1 antibody is chosen from nivolumab, pembrolizumab, cemiplimab, and dostarlimab.

125. A method of treating cancer in a subject in need thereof comprising administering memory NK cells and a multispecific antibody, wherein the multispecific antibody comprises at least a tumor antigen binding moiety and a CD16 binding moiety and is chosen from an NK cell engager or a trispecific killer cell engager.

126. The method of claim 107, wherein the memory NK cells are administered before the antibody.

127. The method of claim 107, wherein the memory NK cells are administered concurrently with the antibody.

128. The method of claim 107, wherein the memory NK cells are administered after the antibody.

129. An immunotherapeutic composition comprising memory NK cells, a multispecific antibody comprising at least a tumor antigen binding moiety and a CD16 binding moiety, and a therapeutically acceptable carrier.

130. An immunotherapeutic kit comprising memory NK cells, a multispecific antibody comprising at least a tumor antigen binding moiety and a CD16 binding moiety, and optionally, instructions to administer the memory NK cells and antibody in simultaneous or sequential combination to a subject with cancer.


SEQUENCE LISTING (60 sequences)

SEQ ID NO: 1 - protein, 485 aa, synthetic construct
DCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEHSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 198 aa, synthetic construct
QGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSITCPPPMSVEHADIWVKSYSLYSRERYICNSGFKRKAGTSSLTECVLNKATNVAHWTTPSLKCIR

SEQ ID NO: 3 - protein, 11 aa, synthetic construct
LASQTIDTWLA

SEQ ID NO: 4 - protein, 7 aa, synthetic construct
AATNLAD

SEQ ID NO: 5 - protein, 9 aa, synthetic construct
QQVYSSPFT

SEQ ID NO: 6 - protein, 23 aa, synthetic construct
DIQMTQSPASLSASVGDRVTITC

SEQ ID NO: 7 - protein, 15 aa, synthetic construct
WYLQKPGKSPQLLIY

SEQ ID NO: 8 - protein, 32 aa, synthetic construct
GVPSRFSGSGSGTDFSFTISSLQPEDFATYYC

SEQ ID NO: 9 - protein, 10 aa, synthetic construct
FGQGTKLEIK

SEQ ID NO: 10 - protein, 106 aa, synthetic construct
DIQMTQSPASLSASVGDRVTITCLASQTIDTWLAWYLQKPGKSPQLLIYAATNLADGVPSRFSGSGSGTDFSFTISSLQPEDFATYYCQQVYSSPFTGQGTKLEIK

SEQ ID NO: 11 - protein, 5 aa, synthetic construct
DYNVY

SEQ ID NO: 12 - protein, 17 aa, synthetic construct
YIDPYNGITIYDQNFKG

SEQ ID NO: 13 - protein, 17 aa, synthetic construct
YIDPYNGITIYDQNLKG

SEQ ID NO: 14 - protein, 8 aa, synthetic construct
DVTTALDF

SEQ ID NO: 15 - protein, 30 aa, synthetic construct
QIQLVQSGGEVKKPGASVRVSCKASGYSFT

SEQ ID NO: 16 - protein, 14 aa, synthetic construct
WVRQSPGKGLEWIG

SEQ ID NO: 17 - protein, 32 aa, synthetic construct
KATLTVDKSTSTAYMELSSLRSEDTAVYFCAR

SEQ ID NO: 18 - protein, 11 aa, synthetic construct
WGQGTTVTVSS

SEQ ID NO: 19 - protein, 117 aa, synthetic construct
QIQLVQSGGEVKKPGASVRVSCKASGYSFTDYNVYWVRQSPGKGLEWIGYIDPYNGITIYDQNFKGKATLTVDKSTSTAYMELSSLRSEDTAVYFCARDVTTALDFWGQGTTVTVSS

SEQ ID NO: 20 - protein, 117 aa, synthetic construct
QIQLVQSGGEVKKPGASVRVSCKASGYSFTDYNVYWVRQSPGKGLEWIGYIDPYNGITIYDQNLKGKATLTVDKSTSTAYMELSSLRSEDTAVYFCARDVTTALDFWGQGTTVTVSS

SEQ ID NO: 21 - protein, 107 aa, synthetic construct [sequence omitted]

SEQ ID NO: 22 - protein, 381 aa, synthetic construct
RTVAAPSVFIFPPSDEQLKSGTASVVCLLNNFYPREAKVQWKVDNALQEFASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGCFYPSDIAVEWESNGQPENNYKTTPPVLDSDGSFFLYSKLTVDKSRWQQGNVFSCSVMHEALHNHYTQKSLSLSPGK

SEQ ID NO: 23 - protein, 474 aa, synthetic construct
YFGKLESKLSVIRNLNMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDSGTTNTVAAYNLTWKSTNFKTILEWEPKPVNQVYTVQISTKSGDWKSKCFYTTDTECDLTDEIVKDVKQTYLARVFSYPAGNVESTGSAGEPLYENSPEFTPYLETNLGQPTIQSFEQVGTKVNVTVEDERTLVRRNNTFLSLRDVFGKDLIYTLYYWKSSSSGKKTAKTNTNEFLIDVDKGENYCFSVQAVIPSRTVNRKSTDSPVECMGQEKGEFRENWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 24 - protein, 583 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGSGGGGSGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASITCPPPMSVEHADIWVKSYSLYSRERYICNSGFKRKAGTSSLTECVLNKATNVAHWTTPSLKCIR

SEQ ID NO: 25 - protein, 5 aa, synthetic construct
NYGVH

SEQ ID NO: 26 - protein, 16 aa, synthetic construct
VIWSGGNTDYNTPFTS

SEQ ID NO: 27 - protein, 11 aa, synthetic construct
ALTYYDYEFAY

SEQ ID NO: 28 - protein, 11 aa, synthetic construct
RASQSIGTNIH

SEQ ID NO: 29 - protein, 7 aa, synthetic construct
YASESIS

SEQ ID NO: 30 - protein, 9 aa, synthetic construct
QQNNNWPTT

SEQ ID NO: 31 - protein, 449 aa, synthetic construct
QVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSAASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 214 aa, synthetic construct
DILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 33 - protein, 7 aa, synthetic construct
SGDYYWT

SEQ ID NO: 34 - protein, 16 aa, synthetic construct
HIYYSGNTNYNPSLKS

SEQ ID NO: 35 - protein, 8 aa, synthetic construct
RVTGAFDI

SEQ ID NO: 36 - protein, 11 aa, synthetic construct
QASQDISNYLN

SEQ ID NO: 37 - protein, 7 aa, synthetic construct
DASNLET

SEQ ID NO: 38 - protein, 9 aa, synthetic construct
QHFDHLPLA

SEQ ID NO: 39 - protein, 221 aa, synthetic construct
QVQLQESGPGLVKPSETLSLTCTVSGGSVSSGDYYWTWIRQSPGKGLEWIGHIYYSGNTNYNPSLKSRLTISIDTSKTQFSLKLSSVTAADTAIYYCVRDRVTGAFDIWGQGTMVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKC

SEQ ID NO: 40 - protein, 214 aa, synthetic construct
DIQMTQSPSSLSASVGDRVTITCQASQDISNYLNWYQQKPGKAPKLLIYDASNLETGVPSRFSGSGSGTDFTFTISSLQPEDIATYFCQHFDHLPLAFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 41 - protein, 5 aa, synthetic construct
DTYIH

SEQ ID NO: 42 - protein, 17 aa, synthetic construct
RIYPTNGYTRYDPKFQD

SEQ ID NO: 43 - protein, 11 aa, synthetic construct
WGGDGFYAMDY

SEQ ID NO: 44 - protein, 11 aa, synthetic construct
KASQDVNTAVA

SEQ ID NO: 45 - protein, 7 aa, synthetic construct
SASFRYT

SEQ ID NO: 46 - protein, 9 aa, synthetic construct
QQHYTTPPT

SEQ ID NO: 47 - protein, 450 aa, synthetic construct
QVQLQQSGPELVKPGASLKLSCTASGFNIKDTYIHWVKQRPEQGLEWIGRIYPTNGYTRYDPKFQDKATITADTSSNTAYLQVSRLTSEDTAVYYCSRWGGDGFYAMDYWGQGASVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSRDELTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 48 - protein, 214 aa, synthetic construct
DIVMTQSHKFMSTSVGDRVSITCKASQDVNTAVAWYQQKPGHSPKLLIYSASFRYTGVPDRFTGSRSGTDFTFTISSVQAEDLAVYYCQQHYTTPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 5 aa, synthetic construct
SYIMM

SEQ ID NO: 50 - protein, 11 aa, synthetic construct
SIYPSGGITFY

SEQ ID NO: 51 - protein, 11 aa, synthetic construct
IKLGTVTTVDY

SEQ ID NO: 52 - protein, 14 aa, synthetic construct
TGTSSDVGGYNYVS

SEQ ID NO: 53 - protein, 7 aa, synthetic construct
DVSNRPS

SEQ ID NO: 54 - protein, 10 aa, synthetic construct
SSYTSSSTRV

SEQ ID NO: 55 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 57 - protein, 451 aa, synthetic construct
QVQLQESGPGLVKPSQTLSLTCTVSGGSISSGDYYWSWIRQPPGKGLEWIGYIYYSGSTDYNPSLKSRVTMSVDTSKNQFSLKVNSVTAADTAVYYCARVSIFGVGTFDYWGQGTLVTVSSASTKGPSVLPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 58 - protein, 214 aa, synthetic construct
EIVMTQSPATLSLSPGERATLSCRASQSVSSYLAWYQQKPGQAPRLLIYDASNRATGIPARFSGSGSGTDFTLTISSLEPEDFAVYYCHQYGSTPLTFGGGTKAEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 59 - protein, 449 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 60 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC